(19) United States
(12) Patent Application Publication
(10) Pub. No.: US 2021/0169854 A1
(43) Pub. Date: Jun. 10, 2021

(54) 3'-[(2Z)-[1-(3,4-dimethylphenyl)-1,5-dihydro-3-methyl-5-oxo-4H-pyrazol-4-ylidene]hydrazino]-2'-hydroxy-[1,1'-biphenyl]-3-carboxylic acid and its salts formulation

(71) Applicant: NOVARTIS AG, Basel (CH)

(72) Inventors: Mangesh Sadashiv Bordawekar, Parsippany, NJ (US); Stephanie Kay Dodd, Ayer, MA (US); Cornelius Stephan Harlacher, Reinach (CH); Jithin K John, Hyderabad (IN); Pravin Karmuse, Hyderabad (IN); Saran Kumar, Edison, NJ (US); Sangeetha Kumari, Hyderabad (IN); Vishnu Maremanda, Andhra Pradesh (IN); Parthkumar Patel, Sathamaba (IN); Kanhaiyalal Patidar, Hyderabad (IN); Kapil Patil, Hyderabad (IN); Alan Edward Royce, Saylorsburg, PA (US); Shaikh Mohsin Shaikh Hamid, Hyderabad (IN); Mauro Serratoni, Basel (CH); Henricus Lambertus Gerardus Maria Tiemessen, Weil am Rhein (DE); Daya D Verma, Edison, NJ (US); Sunita Yadav, Braintree, MA (US)

(21) Appl. No.: 17/025,853

(22) Filed: Sep. 18, 2020

Related U.S. Application Data
(60) Provisional application No. 62/902,992, filed on Sep. 20, 2019.

(51) Int. Cl. A61K 31/4152; A61K 47/22; A61K 9/48; G01N 33/15

(52) U.S. Cl. CPC A61K 31/4152; G01N 33/15; A61K 9/4833; A61K 47/22

(57) ABSTRACT

Disclosed are novel pharmaceutical formulation containing 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid or a pharmaceutically acceptable salt thereof and processes for preparing the same.

### FIELD OF THE INVENTION

The present invention relates to an oral pharmaceutical formulation, suitably tablets, suitably capsules, comprising 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid (INN name eltrombopag) or a pharmaceutically acceptable salt thereof. Suitably the formulation of the present invention comprises eltrombopag monoethanolamine, suitably bis-(monoethanolamine), represented by the following formula (I) and hereinafter referred to as “eltrombopag olamine” or Compound B and at least on micelle or liposome or microemulsion forming agent.

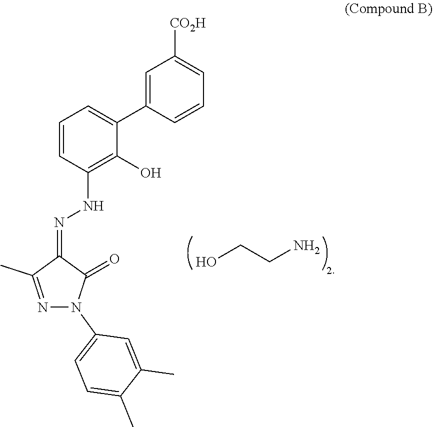

### BACKGROUND OF THE INVENTION

3′-{N′-[1-(3,4-dimethylphenyl)-3-methyl-5-oxo-1,5-dihydropyrazol-4-ylidene]hydrazino}-2′-hydroxybiphenyl-3-carboxylic acid (hereinafter Compound A) is a compound which is disclosed, along with pharmaceutically acceptable salts, hydrates, solvates and esters thereof, as being useful as an agonist of the TPO receptor, particularly in enhancing platelet production and particularly in the treatment of thrombocytopenia, in WO 01/89457, the disclosure of which is hereby incorporated by reference.

Compound eltrombopag bis-(monoethanolamine) is disclosed in WO 03/098002; the disclosure of which is hereby incorporated by reference.

Eltrombopag (US brand Promacta, EU brand Revolade) is currently marketed globally for chronic immune (idiopathic) thrombocytopenia (ITP) and severe aplastic anemia. Both the US and the EU current drug labels contain instructions on avoidance of taking the drug together with food, or closely before or after meal, especially calcium-rich food, for example diary products. For example on the US label it states “Take PROMACTA on an empty stomach (1 hour before or 2 hours after a meal). Take PROMACTA at least 2 hours before or 4 hours after other medications (e.g., antacids), calcium-rich foods. Similarly on the EMA approved Revolade it states “The tablets should be taken at least two hours before or four hours after any products such as antacids, dairy products (or other calcium containing food products), or mineral supplements containing polyvalent cations (e.g. iron, calcium, magnesium, aluminium, selenium and zinc)”

As reported in Promacta drug label, an open-label, randomized-Sequence, crossover trial was conducted to assess the effect of food on the bioavailability of eltrombopag. A standard high-fat breakfast significantly decreased plasma eltrombopag AUCO-∞ by approximately 59% and Cmax by 65% and delayed Tmax by 1 hour. The calcium content of this meal may have also contributed to this decrease in exposure. In a second trial, administration of a single 25-mg dose of eltrombopag for oral suspension to adults with a high-calcium, moderate-fat, moderate-calorie meal reduced plasma eltrombopag AUCO-∞ by 75% (90% CI 71%, 88%) and Cmax by 79% (90% CI: 76%, 82%). Administration of a single 25-mg dose of eltrombopag for oral suspension 2 hours after the high-calcium meal reduced plasma eltrombopag AUCO-∞ by 47% (90% CI: 40%, 53%) and Cmax by 48% (90% CI: 40%, 54%). Administration of a single 25-mg dose of eltrombopag for oral suspension 2 hours before the high-calcium meal reduced plasma eltrombopag AUCO-∞ by 20% (90% CI: 9%, 29%) and Cmax by 14% (90% CI: 2%, 25%).

This is because eltrombopag chelates with coordinating metals, especially with calcium, and forms insoluble complex. As a result eltrombopag formulation has greatly reduced dissolution rate in the presence of calcium (example 8, WO/2008/136843). Hence eltrombopag has reduced bioavailability in the presence of food, especially calcium-rich food (negative food effect).

Generally, negative food effect is associated with BCS Class III drugs (high solubility and poor permeability; Reference: https://cuvillier.de/de/shop/publications/6557). For such poorly permeable drugs, absorptive transporter effects predominate and in presence of food the transporters are inhibited leading to negative food effect. However eltrombopag is low soluble/medium to high permealble. The mechanism for the negative food effect is predominantly related to the property of the drug that is, its tendency to chelate with polyvalent cations. It should be noted that the drop in bioavailability is seen only when the meals are fortified with high levels of calcium while there is negligible drop with low levels of calcium (Daphne D. Williams et. al. Clinical Therapeutics/Volume 31, Number 4, 2009).

Solubility enhancement with surfactant is generally used as means of mitigating positive food effect of poorly soluble drugs. For example the bioavailability of abiraterone increases with food. After a low fat meal, Cmax and AUC are elevated 7- and 5-fold compared with the fasted state, whereas after a high fat meal there is a 17- and 10-fold elevation. W2013/164473 teaches to mitigate food effect by including in the abiraterone formulation one or more lipid excipients and “The majority of these lipid excipients also have surfactant characteristics and many function to improve both the solubility and permeability of abiraterone”. Surfactant does not seem to be an effective means to address negative food effect issue, especially when the food effect is not due to low solubility of the compound, rather due to the complexing property of the compound with polyvalent cations, especially calcium in the meal.

### BRIEF DESCRIPTION OF THE DRAWINGS

Dissolution tests were performed according to Example 4 and some of the results were shown in the figures below.

FIG. 1: Comparison of ETB115 DS, Promacta tablets and Vit E TPGS formulation (formulation 1 in 75 mg) in the presence of 427 mg or 450 mg of calcium or control (absence of calcium), following the dissolution test described in EXAMPLE 4.

FIG. 2A: Comparison of ETB115 DS in different drug load with Vit E TPGS formulation (formulation 2 and 3 in 75 mg) in the presence of 427 mg of calcium or control (absence of calcium) FIG. 2B: Comparison of ETB115 DS in lower drug load (6%) with Vit E TPGS formulation (formulation 12) in the presence of 427 mg of calcium or control (absence of calcium).

FIG. 3A: Dissolution in presence of different concentration of Vit E TPGS in MOPS buffer calcium added 30 min before (FIG. 3A) or 60 minutes after (FIG. 3B) Promacta tablets in dissolution bowl.

FIG. 4A: ETB115 DS with RH40 (formulation 9 in 50 mg) in the presence of 427 mg of calcium or control (absence of calcium)

FIG. 4B: ETB115 DS with MEPC 7 (formulation 11) in the presence of 50 mg or 450 mg of calcium or control (absence of calcium)

FIG. 4C: ETB115 DS with Gellucire 48/16 in the presence of 427 mg of calcium or control (absence of calcium)

FIG. 4D: ETB115 DS with MEPC 3 (formulation 15) in the presence of calcium or control (absence of calcium) Effect of surfactants with increasing value of HLB on the ETB115 dissolution in the absence (FIG. 5A) and presence of 427 mg calcium (FIG. 5B)

FIG. 6 Dissolution tests of lipid formulations of F2, F3 and F4

FIG. 7 Pampa test. ETB115 dissolution and permeation assay indicated as Fasted+High Calcium/Fasted flux ratio.

FIG. 8 Amphiphilicity and functionality expressed on the HLB scale

### DESCRIPTION OF THE INVENTION

Surprisingly we have found that surfactant vitamin E TPGS can effectively mitigate the food effect on eltrombopag, i.e. mitigate the reduction of bioavailability in the presence of food, especially calcium rich food. We have found that vitamin E TPGS can effectively
a) prevent eltrombopag from binding to polyvalent cations (e.g. calcium);
b) prevent the formation of insoluble complex of eltrombopag and polyvalent cations (e.g. calcium);
c) release soluble eltrombopag into the medium despite the presence of excessive amount of polyvalent cations (e.g. calcium), typically in an in vitro dissolution test;
d) increase the solubilization of eltrombopag, typically in an in vitro dissolution test; and/or
e) prevent soluble eltrombopag from crashing out of the medium, typically despite the presence of excessive amount of polyvalent cations (e.g. calcium), typically in an in vitro dissolution test.

Any one of the above effects or any combination thereof can be generally called the anti-calcium effect in this application.

Thus the present invention relates to a pharmaceutical composition (pharmaceutical composition of the invention), preferably in an oral dosage form, comprising Compound 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid (eltrombopag) or a pharmaceutically acceptable salt thereof and Vitamin E TPGS.

In one embodiment the pharmaceutically acceptable salt is monoethanolamine salt. In one embodiment the ratio between eltrombopag and monoethanolamine is 1:1. In one embodiment the ratio between eltrombopag and monoethanolamine is 1:2 (bis-(monoethanolamine)).

In one embodiment the pharmaceutical composition of the invention comprises eltrombopag bis-(monoethanolamine).

Other pharmaceutically acceptable salts include but not limited to sodium salt, potassium salt, magnesium salt, ammonia salt, choline salt, N-methyl-D-glucamine salt, 4-(2-hydroxyethyl)morpholine salt, triethanolamine salt, L-lysine salt, piperazine salt, ethylenediamine salt, diethanolamine salt, N,N′-dimethylethanolamine salt, N,N′-dibenzylethylenedamine salt, tert-butylamine salt, tris(hydroxymethyl) amino methane (also named as tromethamine) salt, 1-(2-hydroxyethyl)pyrrolidine salt and diethylenetriamine salt.

Compound is either in the free acid form or in a pharmaceutically acceptable salt form. Eltrombopag is the INN name of 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid (also named as Compound A). The term “weight of eltrombopag” as used in the context of the present invention refers to the weight of the free acid form, namely Compound A or eltrombopag, unless specified otherwise. For example, 127.5 mg of Compound B should be converted to 100 mg of Compound A when calculating the “weight of eltrombopag”.

In one embodiment the pharmaceutical composition of the invention comprises eltrombopag or a pharmaceutically acceptable salt thereof and vitamin E TPGS, wherein the weight of eltrombopag is not more than 80%, not more than 60%, suitably not more than 40%, suitably not more than 30%, suitably not more than 25%, suitably not more than 20% of the total weight of eltrombopag and vitamin E TPGS. In one embodiment the weight of eltrombopag is not more than 30% of the total weight of eltrombopag and vitamin E TPGS.

By way of example, formulation 1 in Example 1 consists of 95.6 mg of eltrombopag bis-monoethanolaime, corresponding to 75 mg of eltrombopag, and 382 mg of vitamin E TPGS, then the weight of eltrombopag of the total weight of eltrombopag and vitamin E TPGS is 16.4% (75/(75+382)).

In one embodiment the weight of eltrombopag is at least 2%, suitably at least 4%, suitably at least 5%, suitably at least 10% of the total weight of eltrombopag and vitamin E TPGS. In one embodiment the weight of eltrombopag is at least 4% of the total weight of eltrombopag and vitamin E TPGS.

In one embodiment the pharmaceutical composition comprises eltrombopag or a pharmaceutically acceptable salt thereof and vitamin E TPGS, wherein the weight of eltrombopag is from 2% to 50%, suitably from 4% to 30%, suitably from 5% to 25%, suitably from 5 to 20% of the total weight of eltrombopag and vitamin E TPGS.

In one embodiment vitamin E TPGS is the only surfactant in the pharmaceutical composition of the invention.

In one embodiment the pharmaceutical composition of the invention, preferably in an oral dosage form, consists essentially of or consists of eltrombopag or a pharmaceutically acceptable salt thereof and vitamin E TPGS.

Besides vitamin E TPGS, additional surfactants and/or lipids could be added to the pharmaceutical composition of the invention. Typically one or two additional surfactant could be added. Alternatively or additionally one or two lipid could be added. Typically one additional surfactant could be added. Alternatively or additionally one lipid could be added. In one embodiment the pharmaceutical composition of the invention comprises eltrombopag or a pharmaceutically acceptable salt thereof, vitamin E TPGS, Span 80, miglyol 812N, Labrasol.

In one embodiment the pharmaceutical composition of the invention comprises eltrombopag or a pharmaceutically acceptable salt thereof, vitamin E TPGS and at least one more pharmaceutically acceptable excipients.

In one embodiment the at least one more pharmaceutically acceptable excipients include an anti-oxidant. In one embodiment the weight of the anti-oxidant is not more than 10%, suitably not more than 7%, suitably not more than 5%, suitably not more than 3%, suitably not more than 1%, of the total weight of the pharmaceutical composition. Preferably there is only one anti-oxidant in the composition. In one embodiment the anti-oxidant is selected from the list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture thereof, suitably 2 of the anti-oxidants from the list, preferably only one anti-oxidants from the list. In one embodiment the anti-oxidant is vitamin E. In one embodiment vitamin E is not more than 15%, suitably not more than 7%, suitably not more than 5% of the total weight of the pharmaceutical composition. In one embodiment vitamin E is 2-15%, suitably 2-10%, suitably 5% of the total weight of the pharmaceutical composition. In one embodiment the anti-oxidant is BHT. In one embodiment BHT is not more than 3%, suitably not more than 1%, suitably not more than 0.5%, suitably not more than 0.2%, suitably not more than 0.1% of the total weight of the pharmaceutical composition. In one embodiment the anti-oxidant is BHA. In one embodiment BHA is not more 3%, suitably not more than 1%, suitably not more than 0.5%, suitably not more than 0.2% of the total weight of the pharmaceutical composition. In one embodiment the anti-oxidant is Propyl gallate. In one embodiment Propyl gallate is not more than 3%, suitably not more than 1%, suitably not more than 0.5% of the total weight of the pharmaceutical composition. In one embodiment the anti-oxidant is EDTA. In one embodiment weight of EDTA is not more than 10%%, suitably not more than 5%, suitably not more than 2% of the total weight of the pharmaceutical composition. In one embodiment EDTA is 1-5%, suitably 1-3%, suitably 2% of the total weight of the pharmaceutical composition. In one embodiment EDTA is in the form of disodium salt and the weight of EDTA is calculated based on the weight of EDTA disodium.

By way of example, formulation 5 in Example 2 consists of 95.6 mg of eltrombopag bis-monoethanolaime, corresponding to 75 mg of eltrombopag, 358.2 mg of vitamin E TPGS and 23.8 mg of vitamin E, then the weight of eltrombopag of the total weight of eltrombopag and vitamin E TPGS is 17.3% (75/(75+358.2)). In contrast, % w/w, As used in the tables of the Examples, indicates each components' weight percentage of the weight of the total composition. For example ETB115 95.6 mg is the 20% of eltrombopag bis-olamine of a total of 477.6 mg of the composition (drug load, 95.6/477.6=20%). The weight of the anti-oxidant of the total weight of the pharmaceutical composition is 5.0% (23.8/477.6=5%).

In one embodiment the pharmaceutical composition consists essentially of or consists of eltrombopag or a pharmaceutically acceptable salt thereof, vitamin E TPGS and at least one anti-oxidant. In one embodiment the pharmaceutical composition consists of eltrombopag, vitamin E TPGS and only one anti-oxidant. In one embodiment the anti-oxidant is not more than 10%, suitably not more than 7%, suitably not more than 5%, of the total weight of the pharmaceutical composition. In one embodiment the anti-oxidant is selected from a list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture of.

There exists trace amount of vitamin E (about 1.5%) in vitamin E TPGS of GMP standard. This amount of vitamin E is generally regarded as impurities unless otherwise specified in this application.

In one preferred embodiment the anti-oxidant is EDTA.

In one embodiment the pharmaceutical composition comprises eltrombopag or a pharmaceutically acceptable salt thereof, vitamin E TPGS and EDTA. In one embodiment the pharmaceutical composition consists essentially of or consists of eltrombopag, vitamin E TPGS and EDTA.

Without wishing to be bound by the theory, the effect of vitamin E TPGS could be attributed to partial solubilization of eltrombopag in Vitamin E TPGS, which upon contact with aqueous media forms micelles and thus minimizes the interaction of polyvalent cations, e.g. calcium, with the drug.

Eltrombopag, even in the form of bis-monoethanolaime salt, has low solubility in water as well as in a number of liquid/semisolid surfactants (data not shown). However it has been found that other surfactants/lipids also exhibit anti-calcium effect. Without wishing to be bound by the theory, this anti-calcium effect could be plausibly attributed to solubilization or partial solubilization of eltrombopag in such surfactants/lipids containing formulation, which upon contact with aqueous media forms colloids or vesicles, such as micelles or liposomes or microemulsion, and thus minimizes the interaction of polyvalent cations, e.g. calcium, with the drug.

Thus the present invention relates to a pharmaceutical composition (pharmaceutical composition of the invention), preferably in an oral dosage form, comprising eltrombopag or a pharmaceutically acceptable salt thereof and at least one colloid or vesicle forming agent. The term “vesicle” or “colloid”, as used here, can be broadly understood as spherical or non-spherical structures formed by amphiphilic molecules in an aqueous medium. The term “at least one colloid or vesicle forming agent” includes at least one micelle or liposome or microemulsion forming agent. Thus, the pharmaceutical composition of the invention comprises eltrombopag or a pharmaceutically acceptable salt thereof and at least one micelle or liposome or microemulsion forming agent. A micelle forming agent, liposome or microemulsion forming agent are not mutually exclusive. Some agents can form either micelle or liposome or microemulsion depending on the process or on the presence of other components in the medium.

Micelle is generally understood as a spherical structure with diameter about 1 to about 50 nm, about 1 to about 30 nm, about 1-about 20 nm, about 1-about 10 nm, formed with a single layer of amphiphilic molecules with the hydrophilic head directing towards the aqueous phase outside and the lipophilic tails forming a lipophilic compartment at the inside. Liposome is generally a bigger spherical structure with diameter about 30 to about 10000 nm, formed by one or more lipid bilayer(s) surrounding an aqueous inner core. Suitably the vesicle of the present invention is in the size range of about 1-about 5000 nm, about 1-about 3000 nm, about 1-about 1000 nm, about 5-about 500 nm, about 5-about 100 nm, about 5-about 30 nm. The term colloidal-particle is understood as particles in the size range of about 5 to 10000 nm which could be non-spherical and could be single layer or more layers. These colloidal-particles could comprise of polymers alone or in combination of surfactants and lipids. Microemulsion is generally understood as dispersed droplets in the size range of about 10 to about 10000 nm, oily droplets stabilized by a surfactant layer.

The term “at least one micelle or liposome forming agent” as used here, refers to an amphiphilic molecule that is capable of forming micelles or liposomes in an aqueous medium. Typically the concentration of the at least one micelle or liposome forming agent comprised by the pharmaceutical composition of the present invention is above the critical micelle concentration (CMC) or above the critical liposome concentration (CLC), suitably CMC or CLC at 37±0.5 C° in an aqueous medium or in water, upon release from the composition into the medium. Suitably the aqueous medium is gastric fluid or gastric fluid simulates. Suitably the aqueous medium is small intestine fluid or small intestine fluid simulates. Typically the micelles or liposomes or microemulsions formed by the at least one micelle or liposome or microemulsion forming agent of the invention is capable of preventing or partially prevention the interaction of eltrombopag and the polyvalent cations, e.g. calcium, present in the medium.

The in vitro dissolution test as described in EXAMPLE 4 is an easy and effective method of screening suitable micelle or liposome or microemulsion forming agents for the purpose of the present invention. Vitamin E TPGS was selected through this dissolution test as effective in mitigating the calcium effect on eltrombopag. Such effect was further confirmed in the Macroflux tests (PAMPA assays EXAMPLE 8).

A micelle/liposome/microemulsion forming agent for the purpose of the present invention should be pharmaceutically acceptable. Regulatory health authorities provide guidance of pharmaceutically acceptable excipients (e.g. https://www.accessdata.fda.gove/scripts/cder/lig/index.cfm). Furthermore the minimum concentration needed for a particular micelle forming agent for the purpose of the invention, e.g. above CMC in aqueous medium, should not exceed its maximum amount as set forth by the regulatory health authorities. For example 764 mg of Vitamin E TPGS is the maximum daily amount allowed for children older than one 1 year.

In one embodiment the present invention relates to a pharmaceutical composition (pharmaceutical composition of the invention), preferably in an oral dosage form, comprises eltrombopag or a pharmaceutically acceptable salt thereof and phospholipids. Preferably the phospholipids are pharmaceutically acceptable.

Phospholipids are surface-active, amphiphilic molecules, which comprise a polar head group and a lipophilic tail. The diacyl-phospholipids (DA-PL) comprise a glycerol backbone, which is esterified in positions 1 and 2 with fatty acids and in position 3 with phosphate, whereas phospholipids with one fatty acid tail are called “monoacyl-phospholipids” (MA-PL) or “lyso-phospholipids”. In typical membrane phospholipids, the phosphate group is further esterified with an additional alcohol, for instance in phosphatidylcholine (PC) with choline, in phosphatidylethanolamine (PE) with ethanolamine, and in phosphatidylglycerol (PG) with glycerol. Depending upon the structure of the polar region and pH of the medium, PE and PC are zwitterionic and have a neutral charge at pH values of about 7, whereas PG is negatively charged. The most common phospholipid is PC, and PC is the main component of lecithin.

Lecithin is described, e.g., in the United States Pharmacopoeia (USP) as a “complex mixture of acetone-insoluble phosphatides, which consists chiefly of PC, PE, phosphatidylserine, and phosphatidylinositol, combined with various amounts of other substances such as triglycerides, fatty acids, and carbohydrates, as separated from the crude vegetable oil source. It contains not less than 50% of acetone-insoluble matter.” Normally, lecithin grades containing more than 80% PC do not comply anymore with the phamacopoeial definition and are called arbitrarily PC, whereas grades containing less than 80% PC can be arbitrarily called lecithin.

As understood by a skilled person, phospholipids, normally extracted from natural sources, is a mixture in which DA-PL is the predominant species over MA-PL. DA-PL is further a mixture with different phosphatidyl derivatives and different length and saturation of the fatty acids. Here below the two tables indicate the composition of lecithin obtained from different natural sources. Although phospholipids can be chemically synthesized, it is cheaper and environmental friendlier to obtain phospholipids fromnatural sources.

TABLE 1
Phospholipid composition of vegetable de-oiled lecithins,
as derived from corresponding product specifications (%)

| | Lecithin | | |
|---|---|---|---|
| | | Sunflower | |
| Phospholipid | Soybean | seed | Rapeseed |
| PC | 20-22 | 20-26 | 23-31 |
| PE | 16-22 | 4-10 | 9-15 |
| PI | 13-16 | 15-19 | 15-18 |
| PA | 5-10 | 2-5 | 5-10 |
| LPC | <3 | <3 | <3 |

LPC: lyso-phosphotidyl choline

TABLE 2
Fatty acid composition of typical batches
of vegetable deoiled lecithins (area %)

| | Lecithin | | |
|---|---|---|---|
| | | Sunflower | |
| Fatty add | Soybean | seed | Rapeseed |
| C14:0 | 0.1 | 0.1 | 0.1 |
| C16:0 | 21 | 16 | 10 |
| C18:0 | 4.7 | 5.3 | 0.8 |
| C18:1 | 9.9 | 21 | 49 |
| C18:2 | 57 | 54 | 31 |
| C18:3 | 5.0 | 0.2 | 4.4 |
| C20:0 | 0.1 | 0.3 | 0.1 |
| C22:0 | 0.4 | 15 | 0.1 |

In one embodiment the phospholipids is diacyl-phospholipids.

In one embodiment the phospholipids is lecithin.

The company Lipoid (https://www.lipoid.com/en/node/10) produces a big variety of phospholipids products suitable for the present invention, which includes but not limited to Lipoid 16:1/18-1, Lipoid, DMPG NA, Lipoid P 75, Lipoid S 80, Lipoid S, Lipoid R, Lipoid E and Lipoid E PG/DSPG.

Synthetic DA-PLs, such as Lipoid PC, Lipoid PE, Lipoid PG, Lipoid PA, Lipoid PS can also be purchased from Lipoid catalog.

The Company ALC (http://www.amecanlecibin.com/abotphos.html) also offers a variety of phospholipids.

In one embodiment the phospholipids is negative charged. It has been observed that pharmaceutical composition of the present invention comprising negative charged phospholipids exerts stronger anti-calcium effect than neutral charged phospholipids at pH values of about 7. Without wishing to be bound by the theory, negatively charged phospholipids can have the anti-calcium effect by additionally capturing positively charged calcium. Thus in one embodiment the phospholipids is negatively charged. In one embodiment the phospholipids is lecithin. In one embodiment the phospholipids is phosphatidylglycerol.

In aqueous medium diacyl-phospholipids normally form liposomes. The participation of surfactant in the formation of vesicles increases the curvature, which results in liposomes with smaller diameter or results in thermodynamically stable micelles. The addition of surfactant, such as monoacyl-phospholipids or bile salt, reduces the viscosity of the formulation.

Thus in one preferred embodiment, the pharmaceutical composition comprises eltrombopag or a pharmaceutically acceptable salt thereof, diacyl-phospholipids and at least one surfactant, preferably one surfactant. In one embodiment the at least one surfactant is monoacyl-phospholipids. In one embodiment the at least one surfactant is bile salt. In one embodiment the at least one surfactant is vitamin E TPGS.

In one embodiment the pharmaceutical composition comprises diacyl-phospholipids and monoacyl-phospholipids. Typically the molar ratio between monoacyl-phospholipids and diacyl-phospholipids is from at least about 1:20 to up to about 1:4, to up to about 1:3, to up to about 1:2, to up to about 1:1.

In one embodiment the pharmaceutical composition comprises diacyl-phospholipids and at least one bile salt, preferably one bile salt. Typically the molar ratio between diacyl-phospholipids and the bile salt is from about 3:1 to about 1:3, from about 2:1 to about 1:2 and more typically about 1:1.

In one embodiment the pharmaceutical composition comprises diacyl-phospholipids, monoacyl-phospholipids and at least one bile salt, preferably one bile salt.

Suitable bile salts include, but not limited to, sodium cholate, sodium deoxycholate, sodium chenodeoxycholate, sodium lithocholate, sodium ursodeoxycholate, sodium hyodeoxycholate, glycine conjugated sodium glycocholate, sodium glycodeoxycholate, sodium glycochenodeoxycholate, sodium glycoursodeoxycholate, taurine conjugated sodium taurocholate, sodium taurodeoxycholate, sodium taurochenodeoxycholate.

In one embodiment the bile salt is selected from a group consisting of sodium taurocholate, sodium taurodeoxycholate, sodium taurochenodeoxycholate, sodium glycocholate, sodium glycodeoxycholate and sodium glycochenodeoxycholate.

In one embodiment the bile salt is selected from a group consisting of sodium cholate, sodium deoxycholate, sodium glycocholate, sodium taurocholate, and sodium taurodeoxycholate.

In one embodiment the bile salt is sodium taurocholate.

In one embodiment the bile salt is sodium glycocholate.

Alternatively bile salts exist in abundance in gastric intestinal tract, which could interact with the DA-PL released from the composition to form micelles or small sized liposomes even if the composition itself does not comprise bile salt.

In one embodiment the phospholipids is monoacyl-phospholipids. Lipoid LPC 80 contains 70%-80% of monoacyl-phospholipids, while the rest is mainly diacyl-phospholipids. In one embodiment the pharmaceutical composition comprises phospholipids, wherein phospholipids is predominantly lyso-phospholipids. Used in this context, the term “predominantly” is understood that the molar ratio between monoacyl-phospholipids and diacyl-phospholipids is from at least about 1:1 to up to about 2:1, to up to about 3:1, to up to about 4:1, to up to about 5:1.

In one embodiment, the pharmaceutical composition of the invention, comprises eltrombopag or a pharmaceutically acceptable salt thereof, phospholipids and at least one co-solvent. Co-solvent is miscible with water and can increase the solubilization of the drug. Preferably the co-solvent is pharmaceutically acceptable. The minimum concentration needed for the co-solvent for the purpose of the invention should not exceed its maximum amount regulated by the regulatory health authorities. Generally the weight of co-solvent does not exceed 20%, does not exceed 15%, does not exceed 10%, does not exceed 5% of the total weight of the pharmaceutical composition.

Commonly used co-solvent includes but not limited to PEG300, propylene glycol.

In one embodiment the co-solvent is PEG 300.

In one embodiment, the pharmaceutical composition of the invention, comprises eltrombopag or a pharmaceutically acceptable salt thereof, phospholipids and at least one viscosity-lowering agent. Viscosity-lowering agent includes but not limited to glycerol. Generally the weight of co-solvent does not exceed 15%, does not exceed 10%, does not exceed 5% of the total weight of the pharmaceutical composition.

In one embodiment, in the pharmaceutical composition of the invention comprising phospholipids, the weight of eltrombopag, calculated in its free acid form, is not more than 40%, typically and preferably not more than 30%, not more than 20%, not more than 15% of the total weight of the pharmaceutical composition. In one embodiment the weight of eltrombopag, calculated in its free acid form, is not more than 20% of the total weight of the pharmaceutical composition. In one embodiment the weight of eltrombopag, calculated in its free acid form, is between about 3% to about 40%, between about 3% to about 30%, between about 5%-about 25%, preferably between about 5%-about 20% of the total weight of the pharmaceutical composition.

In one embodiment the weight of phospholipids is at least at least 35%, at least 50%, at least 60%, at least 70% of the total weight of the pharmaceutical composition.

In one embodiment the weight of phospholipids is not more than 90% of the total weight of the pharmaceutical composition.

In one embodiment the weight of diacyl-phospholipids is not more than 80%, not more than 60%, not more than 50% of the total weight of the pharmaceutical composition.

In one embodiment the weight of diacyl-phospholipids is between about 35% to about 85%, between about 50% to about 75% of the total weight of the pharmaceutical composition.

In one embodiment, the pharmaceutical composition of the invention, comprises, consists essentially of or consists of
a) About 4-20% w/w eltrombopag or a pharmaceutically acceptable salt thereof, calculated based on eltrombopag free acid; and
b) About 45% w/w to about 94% to 95% w/w of phospholipids.

In one embodiment, the pharmaceutical composition of the invention, comprises, consists essentially of or consists of
c) About 4-20% w/w eltrombopag or a pharmaceutically acceptable salt thereof, calculated based on eltrombopag free acid;
d) About 45% w/w to about 85% w/w of phospholipids; and
e) About 10% to 45% bile salt.

In one embodiment, the pharmaceutical composition of the invention, comprises, consists essentially of or consists of eltrombopag or a pharmaceutically acceptable salt thereof, phospholipids, preferably diacyl-phospholipids, and a bile salt. In one embodiment, the pharmaceutical composition of the invention, comprises, consists essentially of or consists of eltrombopag or a pharmaceutically acceptable salt thereof, phospholipids, preferably diacyl-phospholipids, a bile salt and a viscosity-lowering agent. In one embodiment the viscosity-lowering agent is glycerol. In one embodiment, the pharmaceutical composition of the invention, comprises, consists essentially of or consists of
f) About 4-20% w/w eltrombopag or a pharmaceutically acceptable salt thereof, calculated based on eltrombopag free acid;
g) About 40-80% w/w phospholipids, preferably diacyl-phospholipids;
h) About 10-40% w/w bile salt; and
i) About 0-10% w/w viscosity-lowering agent.

In one embodiment the pharmaceutical composition of the invention, comprises, consists essentially of or consists of eltrombopag or a pharmaceutically acceptable salt thereof, phospholipids, preferably predominately lyso-phospholipids. In one embodiment, the pharmaceutical composition of the invention, comprises, consists essentially of or consists of eltrombopag or a pharmaceutically acceptable salt thereof, phospholipids, preferably predominately lyso-phospholipids and a co-solvent. Preferably the co-solvent is PEG 300.

In one embodiment the pharmaceutical composition of the invention, comprises, consists essentially of or consists of
a) About 5-20% w/w eltrombopag or a pharmaceutically acceptable salt thereof, based on eltrombopag free acid;
b) About 60% to 85% w/w phospholipids, calculated based on the total weight of phospholipids, wherein phospholipids comprises predominately lyso-phospholipids;
c) About 0%-10% w/w co-solvent, preferably the co-solvent is PEG 300.

In one aspect the present invention relates to a method of manufacturing the pharmaceutical composition comprising the steps of mixing eltrombopag or a pharmaceutically acceptable salt thereof and phospholipids in a solution (Solution). Thus the resulting pharmaceutical composition is in the liquid form, which can be administered as oral solutions, as concentrated solutions to be filled in capsules or dosed by pipetting a small volume in a drink like water or juice. Organic solvent in the Solution can be evaporated to result in solid or semi-solid cake. Such cake can be directly formulated into tablets or filled into capsules. Optionally such case can be re-hydrated with water to arrive at a solution, that can be filled into capsules. The capsule is preferably sealed by banding.

Alternatively the pharmaceutical formulation of the present invention comprising phospholipids is in solid form. Typically the Solution can be dried in the presence of a sugar (Van Hoogevest, European Journal of Pharmaceutical Sciences, Vol 108, Page 1-12, 2017). Alternatively, in order to convert unsaturated mono- and diacyl-phospholipids into powders organic (ethanolic) solvent solutions of the phospholipids can be mixed as explained in WO2003063835A with absorbing porous carriers like Neuselin Grade 2 (Fuji Chemicals) and subsequently the solvent is removed under vacuum. Furthermore CA2352178 teaches a method of mixing the phospholipids containing solution with polymers, dry and grind to obtain a free flowing powder.

The present invention relates to a pharmaceutical composition (pharmaceutical composition of the invention), preferably in an oral dosage form, comprising eltrombopag or a pharmaceutically acceptable salt thereof and at least one surfactant.

Suitably the term “at least one surfactant” refers to one, two, three or four surfactants present in the pharmaceutical composition of the present invention. Suitably the term “at least one surfactant” refers to one, two or three surfactants present in the pharmaceutical composition of the present invention. Suitably the term “at least one surfactant” refers to one or two surfactants present in the pharmaceutical composition of the present invention. Suitably the term “at least one surfactant” refers to only one surfactant present in the pharmaceutical composition of the present invention.

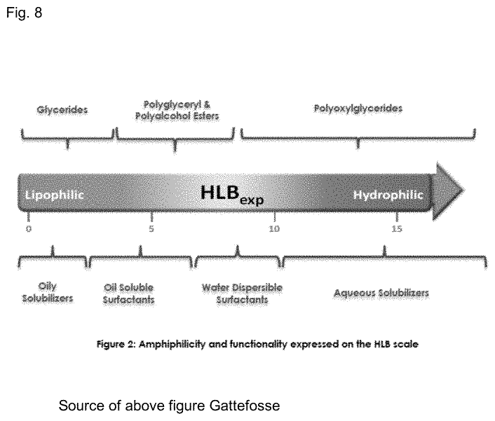

An empirical system known as hydrophilic/lipophilic balance (HLB) is commonly used to categorize amphiphilic surfactants by the degree of affinity for the oily phase compared to aqueous phases in a formulation (see FIG. 8).

The HLB of a surfactant can be determined by analytical methods and referred to as practical HLB. Alternatively the HLB value can be obtained theoretically. For nonionic surfactants the common way to calculate HLB is called Griffin method (Griffin, William C. (1954), “Calculation of HLB Values of Non-Ionic Surfactants” (PDF), Journal of the Society of Cosmetic Chemists, 5 (4): 249, archived from the original (PDF) on 2014-08-12, retrieved 2013-05-25). Practically surfactants providers usually provides information on HLB value. HLB value can vary to certain degree, for example 3, 2 or 1 between different providers or between different batches of the same provider largely due to varying degree of polymerization, e.g. the number of PEG repeats. HLB values of commonly used and/or commercially available surfactants are collected from general literature, including product catalogs and are presented in Table 3.

According to the Gattefosse diagram above, the surfactant suitable for the present invention is typically in the range of water dispersible surfactant, preferably in the range of aqueous solubilizers. Typically eltrombopag is at least partially solubilized by the aid of the at least one surfactant.

The working surfactant examples suggest that surfactant suitable for the present invention is likely more towards the hydrophilic end of the diagram above. Thus in one embodiment the at least one surfactant has a HLB value above 7, about 8, above 9, preferably above 10, more preferably above 11. The HLB value should be viewed with certain degree of flexibility due to the variation range of 3, 2 or 1 in practice. In one embodiment the at least one surfactant has a HLB value below 20, below 18, preferably below 17, more preferably below 16. In one embodiment the at least one surfactant has a HLB value in the range of 9-20, preferably 10-19, preferably 10-18, preferably 11-17, more preferably 12-16.

The surfactant suitable for the present invention is preferably a polyethoxylated/polyethylene glycol/PEG fatty acid ester derivative, such as PEG 40 hydrogenated castor oil (Cremophor RH 40), PEG 35 castor oil (Cremophor EL), PEG 32 monostearate (Gelucire 48/16), PEG 15 hydroxystearate (Solutol HS 15), or Vitamin E TPGS (d-α-tocopheryl PEG 1000 succinate) or mixtures thereof. The respective HLB of about 14-16, 12-14, 12, 14-16, or 13 of the above molecules is related to but not exclusively dependent on the number of ethylene oxide repeat units in the PEG chain.

The below table contains commonly used surfactants with indication of their properties and suitability for the present invention.

TABLE 3
List of Surfactants and Lipids:

| | | | | | | | | useable |
|---|---|---|---|---|---|---|---|---|
| | | | | | | | Pegylation | for |
| Ref | Class/type | Surfactant | HLB | IIG level | | Application | (yes/no) | ETB |
| Chemical book | Non-ionic | Polyethylene Glycol | 15.7 | | | Cosmetic | yes | likely |
| | | Monocetyl Ether | | | | | | |
| | Non-ionic | POLYETHYLENE | 16.9 | | | Cosmetic- | yes | likely |
| | | GLYCOL MONOOLEYL | | | | Shampoo | | |
| | | ETHER | | | | | | |
| | Non-ionic | Glycerol tristearate | 5.8 | 225 | mg | Oral | No | |
| | | | | | | formulation | | |
| | Non-ionic | Sorbitan | 6.7 | 2% | w/w | Topical | No | |
| | | monopalmitate | | | | Formulation | | |
| | Non-ionic | TRIOLEIN | | | | Injectable | No | |
| | | | | | | preparation | | |
| | Non-ionic | Span 20 | 8.6 | 83.9 | mg | Oral | yes | |
| | | | | | | formulation | | |
| | Non-ionic | Span 60 | 4.7 | 3.5 | mg | Oral | yes | |
| | | | | | | formulation | | |
| | Non-ionic | Span 80 | 4.3 | 153.9 | mg | Oral | yes | |
| | | | | | | formulation | | |
| | Non-ionic | Tween 80 | 15 | 418 | mg | Oral | yes | likely |
| | | | | | | formulation | | |
| | Non-ionic | Tween 85 | 11 | | | | yes | likely |
| | Non-ionic | Tween 60 | 15 | 20 mg/1 ml | | Oral | yes | likely |
| | | | | | | emulsion | | |
| | Non-ionic | Polysorbate 20 | 16.7 | 56.25 | mg | Oral | yes | likely |
| | | | | | | formulation | | |
| | Non-ionic | Polyoxyethylene | 18.8 | 25 mg/5 ml | | Oral | yes | yes |
| | | stearate | | | | Concentrate | | |
| | Non-ionic | Glyceryl Monooleate | | | | | no | |
| | Non-ionic | SORBITAN | 1.8 | 1.5 mg/5 ml | | Powder for | no | |
| | | TRIOLEATE | | | | suspension | | |
| | Non-ionic | Polyoxyethylene | water | 5.22% | W/V | Topical | yes | likely |
| | | lauryl ether | soluble | | | Formulation | | |
| | Non-ionic | Propyleneglycol | | 250 | mg | Powder for | no | |
| | | alginate | | | | suspension | | |
| | Non-ionic | GLYCEROL | | | | | no | |
| | | MONOHYDROXY- | | | | | | |
| | | STEARATE | | | | | | |
| | Non-ionic | Fatty acids, lanolin, | 10 | | | Cosmetic | no | |
| | | isopropyl esters | | | | | | |
| | Non-ionic | Poly(ethylene glycol) | | | | Topical | Yes | likely |
| | | distearate | | | | formulation | | |
| | Non-ionic | MYRISTYL | 8.5 | | | Cosmetic | | |
| | | MYRISTATE | | | | | | |
| | Non-ionic | SUCROSE | | | | | | |
| | | DISTEARATE | | | | | | |
| | Non-ionic | SORBITAN | | 2.5% | W/W | Topical | | |
| | | SESQUIOLEATE | | | | formulation | | |
| | Non-ionic | SORBITAN | 2.1 | 0.5% | W/W | Topical | | |
| | | TRISTEARATE | | | | Formulation | | |
| | Non-ionic | glycerine monostearate | | | | | no | |
| | Non-ionic | Fatty alcohol | | | | | YES | likely |
| | | polyoxyethylene ether | | | | | | |
| | | N = 3 | | | | | | |
| | Non-ionic | castor oil polyoxyethylene | | | | | YES | likely |
| | | (90) ether | | | | | | |
| | Non-ionic | MONOMYRISTIN | 11.5 | | | | No | |
| | Non-ionic | alkyl polyglucoside | | | | | No | |
| | Non-ionic | TRIDECETH-4 | | | | cosmetic | | |
| | Non-ionic | MONOCAPRYLIN | 6 | | | | no | |
| | Non-ionic | TRILAURIN | Soluble | | | | no | |
| | | | in water | | | | | |
| | Non-ionic | DILAURIN | 7 | | | | no | |
| | Non-ionic | MONOLAURIN | | | | | no | |
| | Non-ionic | C\hat{ }(8~9\hat{ }) alkyl phenyl | | | | | yes | likely |
| | | polyoxyethylene (15) ether | | | | | | |
| | Non-ionic | C\hat{ }(12~18\hat{ }) fatty alcohol | | | | | yes | likely |
| | | polyoxyethylene (35) ether | | | | | | |
| | Non-ionic | C\hat{ }(8~9\hat{ }) alkyl phenyl | | | | | yes | likely |
| | | polyoxyethylene (8) ether | | | | | | |
| | Non-ionic | alkyl phenyl | | | | | yes | likely |
| | | polyoxyethylene ether | | | | | | |
| | Non-ionic | octyl phenyl | | | | | yes | likely |
| | | polyoxyethylene (30) ether | | | | | | |
| | Non-ionic | dibenzyl biphenyl | | | | | yes | likely |
| | | polyoxyethylene ether | | | | | | |
| | Non-ionic | nonyl phenyl | | | | | yes | likely |
| | | polyoxyethylene (9) ether | | | | | | |
| | Non-ionic | octyl phenyl | | | | | yes | likely |
| | | polyoxyethylene (3) ether | | | | | | |
| | Non-ionic | castor oil poloxyethylene | | | | | yes | likely |
| | | (30) ether | | | | | | |
| | Non-ionic | polyoxyethylene (10) | | | | | yes | likely |
| | | castor oil ether | | | | | | |
| | Non-ionic | DECYL OLEATE | Insoluble | | | | no | |
| | | | in water | | | | | |
| | Non-ionic | Trimethylolpropane t | 2 | | | | no | |
| | Non-ionic | SUCROSE COCOATE | 3 | | | | no | |
| | Non-ionic | CETYL LACTATE | Insoluble | | | Cosmetic | | |
| | | | in water | | | | | |
| | Non-ionic | Sucrose stearate | Slightly | 44.5 | mg | Oral | | |
| | | | soluble | | | formulation | | |
| | | | in water | | | | | |
| | Non-ionic | Isooctyl palmitate | | | | cosmetic | | |
| | Non-ionic | Pentaerythrityl | | | | | | |
| | | tetrastearate | | | | | | |
| | Non-ionic | Isopropyl myristate | | | | Topical | | |
| | | | | | | formulation | | |
| | Non-ionic | Isooctadecanoic acid, ester | | | | | | |
| | | with 1,2,3-propanetriol | | | | | | |
| | Non-ionic | ethylene glycol | | | | | yes | |
| | | monostearate | | | | | | |
| | Non-ionic | HEXAETHYLENE | | | | | yes | |
| | | GLYCOL MONOOCTYL | | | | | | |
| | | ETHER | | | | | | |
| | Non-ionic | glycerine monolaurate | | | | | no | |
| | Non-ionic | 1-Glyceryl caprate | | | | | no | |
| | polymer | Hydroxypropyl methyl | Soluble | 670 | mg | Oral | no | No |
| | | cellulose | in water | | | formulation | | |
| | polymer | Hydroxyethyl Cellulose | | 400 | mg | Oral | no | |
| | | | | | | Formulation | | |
| | polymer | Soluplus ® Polyvinyl | | NA | | | yes | No |
| | | caprolactam-polyvinyl | | | | | | |
| | | acetate-polyethylene | | | | | | |
| | | glycol graft copolymer. | | | | | | |
| Gattefosse | Non-ionic | Gelucire 48/16 HLB 12 | 12 | | | | yes | yes |
| brochure | | (PEG32 stearate or | | | | | | |
| | | Polyoxyl stearate) | | | | | | |
| Gattefosse | Non-ionic | Labrasol ALF | 12 | | | | | yes |
| brochure | | HLB 12 | | | | | | |
| Gattefosse | Non-ionic | Gelucire 44/14 | 11 | 3 mg/218 mg | | | | Very |
| brochure | | HLB 11 | | basis RDS | | | | likely |
| Gattefosse | Non-ionic | Gelucire 50/13 | 11 | | | | | Very |
| brochure | | HLB 11 | | | | | | likely |
| Gattefosse | Non-ionic | Labrafil M 1944 CS | 9 | | | | | |
| brochure | | HLB 9 | | | | | | |
| Gattefosse | Non-ionic | Labrafil M 2125 CS | 9 | | | | | |
| brochure | | HLB 9 | | | | | | |
| Gattefosse | Non-ionic | Labrafil M 2130 CS | 9 | | | | | |
| brochure | | HLB9 | | | | | | |
| Gattefosse | Non-ionic | Glyceryl mono | 3.8 | | | | | |
| brochure | | stearate HLB 11 | | | | | | |
| | Non-ionic | Pluronic ® L-31 | 1 to 7 | NA | | | yes | |
| | | Non-ionic 1100 | | | | | | |
| | | 1.0-7.0 | | | | | | |
| | Non-ionic | Pluronic ® L-35 | 18 to 23 | | | | yes | |
| | | Non-ionic 1900 | | | | | | |
| | | 18.0-23.0 | | | | | | |
| | Non-ionic | Pluronic ® L-61 | 1 to 7 | | | | yes | |
| | | Non-ionic 2000 | | | | | | |
| | | 1.0-7.0 | | | | | | |
| | Non-ionic | Pluronic ® L-81 | 1 to 7 | | | | yes | |
| | | Non-ionic 2800 | | | | | | |
| | | 1.0-7.0 | | | | | | |
| | Non-ionic | Pluronic ® L-64 | 12 to 18 | | | | yes | |
| | | Non-ionic | | | | | | |
| | | 2900 12.0-18.0 | | | | | | |
| | Non-ionic | Pluronic ® L-121 | 1 to 7 | | | | yes | |
| | | Non-ionic 4400/ | | | | | | |
| | | Poloxamer 401 | | | | | | |
| | Non-ionic | Pluronic ® P-123 | 7 to 9 | NA | | | yes | |
| | | Non-ionic 5800/ | | | | | | |
| | | Poloxamer 403 | | | | | | |
| | Non-ionic | Pluronic ® F-68 | >24 | 100 | | | yes | No |
| | | Non-ionic 8400/ | | | | | | |
| | | Poloxamer 188 | | | | | | |
| | Non-ionic | Pluronic ® F-108 | >24 | NA | | | yes | |
| | | Non-ionic 14600/ | | | | | | |
| | | Poloxamer 338 | | | | | | |
| https://patents. | Non-ionic | Brij ®30 Non-ionic | 9.7 | NA | | | yes | likely |
| google.com/patent/ | | Polyoxyethylene (4) | | | | | | |
| KR101841756B1/ | | lauryl ether 9.7 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®35 Non-ionic | 16.9 | NA | | | yes | likely |
| google.com/patent/ | | Polyoxyethylene (23) | | | | | | |
| KR101841756B1/ | | lauryl ether 16.9 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®52 Non-ionic | 5.3 | NA | | | yes | |
| google.com/patent/ | | Polyoxyethylene (2) | | | | | | |
| KR101841756B1/ | | cetyl ether 5.3 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®56 Non-ionic | 12.9 | NA | | | yes | likely |
| google.com/patent/ | | Polyoxyethylene (10) | | | | | | |
| KR101841756B1/ | | Cetyl ether 12.9 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®58 Non-ionic | 15.7 | NA | | | yes | likely |
| google.com/patent/ | | Polyoxyethylene (20) | | | | | | |
| KR101841756B1/ | | Cetyl ether 15.7 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®72 Non-ionic | 4.9 | NA | | | yes | |
| google.com/patent/ | | Polyoxyethylene (2) | | | | | | |
| KR101841756B1/ | | stearyl ether 4.9 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®76 Non-ionic | 12.4 | NA | | | yes | likely |
| google.com/patent/ | | Polyoxyethylene (10) | | | | | | |
| KR101841756B1/ | | stearyl ether 12.4 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®78 Non-ionic | 15.3 | NA | | | yes | likely |
| google.com/patent/ | | Polyoxyethylene (20) | | | | | | |
| KR101841756B1/ | | stearyl ether 15.3 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®92V Non-ionic | 4.9 | NA | | | yes | |
| google.com/patent/ | | Polyoxyethylene (2) | | | | | | |
| KR101841756B1/ | | oleyl ether 4.9 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®93 Non-ionic | 4 | NA | | | yes | |
| google.com/patent/ | | Polyoxyethylene (2) | | | | | | |
| KR101841756B1/ | | oleyl ether 4 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®96V Non-ionic | 4 | NA | | | yes | |
| google.com/patent/ | | Polyethylene glycol | | | | | | |
| KR101841756B1/ | | oleyl ether 12.4 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®97 Non-ionic | 12 | NA | | | yes | likely |
| google.com/patent/ | | Polyoxyethylene (10) | | | | | | |
| KR101841756B1/ | | oleyl ether 12 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®98 Non-ionic | 15.3 | NA | | | yes | likely |
| google.com/patent/ | | Polyoxyethylene (20) | | | | | | |
| KR101841756B1/ | | oleyl ether 15.3 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Brij ®700 Non-ionic | 18 | NA | | | yes | likely |
| google.com/patent/ | | Polyoxyethylene (100) | | | | | | |
| KR101841756B1/ | | stearyl ether 18 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®20 Non-ionic | 8.6 | 83.9 | | | No | |
| google.com/patent/ | | Sorbitan monolaurate | | | | | | |
| KR101841756B1/ | | 8.6 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®40 Non-ionic | 6.7 | NA | | | No | |
| google.com/patent/ | | Sorbitan monopalmitate | | | | | | |
| KR101841756B1/ | | 6.7 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®60 Non-ionic | 4.7 | 62.5 | | | No | |
| google.com/patent/ | | Sorbitan monostearate | | | | | | |
| KR101841756B1/ | | 4.7 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®65 Non-ionic | 2.1 | NA | | | No | |
| google.com/patent/ | | Sorbitan tristearate | | | | | | |
| KR101841756B1/ | | 2.1 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®80 Non-ionic | 4.3 | 153.9 | | | No | |
| google.com/patent/ | | Sorbitan monooleate | | | | | | |
| KR101841756B1/ | | 4.3 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Span ®85 Non-ionic | 1.8 | 1.5 mg/5 ml | | | No | |
| google.com/patent/ | | Sorbitan trioleate 1.8 | | | | | | |
| KR101841756B1/ | | | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Myrj ®45 Non-ionic | 10.8 | NA | | | yes | likely |
| google.com/patent/ | | Polyoxyethylene | | | | | | |
| KR101841756B1/ | | monostearate | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Myrj ®49 Non-ionic | 15 | NA | | | yes | likely |
| google.com/patent/ | | Polyoxyethylene | | | | | | |
| KR101841756B1/ | | monostearate | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Myrj ®52 Non-ionic | 16.9 | NA | | | yes | likely |
| google.com/patent/ | | Polyoxyethylene | | | | | | |
| KR101841756B1/ | | monostearate | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Myrj ®53 Non-ionic | 17.9 | NA | | | yes | likely |
| google.com/patent/ | | Polyoxyethylene | | | | | | |
| KR101841756B1/ | | monostearate | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Kolliphor EL/ELP - | | 599.4 | mg | | yes | yes |
| google.com/patent/ | | Macrogolglycerol | | | | | | |
| KR101841756B1/ | | ricinoleate | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Polyoxyethylene (20) | 16.7 | 56.25 | | | yes | likely |
| google.com/patent/ | | sorbitan monolaurate - | | | | | | |
| KR101841756B1/ | | Tween 20 HLB 16.7 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Polyoxyethylene (20) | 15.6 | 0.05 mg/ ml | | | yes | likely |
| google.com/patent/ | | sorbitan monopalmitate - | | | | | | |
| KR101841756B1/ | | Tween 40 HLB 15.6 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Polyoxyethylene (20) | 15 | 25 mg/5 ml | | | yes | likely |
| google.com/patent/ | | sorbitan monostearate - | | | | | | |
| KR101841756B1/ | | Tween 60 HLB 15 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| https://patents. | Non-ionic | Polyoxyethylene (20) | 15 | 418.37 | | | yes | likely |
| google.com/patent/ | | sorbitan mono-oleate - | | | | | | |
| KR101841756B1/ | | Tween 80 HLB 15 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| Handbook of | Non-ionic | Polyoxyethylene (20) | 10.5 | NA | | | yes | likely |
| excipient for HLB | | sorbitan tristearate - | | | | | | |
| | | Tween 65 | | | | | | |
| https://patents. | Non-ionic | Polyoxyethylene (20) | 11 | NA | | | yes | likely |
| google.com/patent/ | | sorbitan tri-oleate - | | | | | | |
| KR101841756B1/ | | Tween 85 HLB 11 | | | | | | |
| en? [symbol] =non- | | | | | | | | |
| ionic+surfactant+ | | | | | | | | |
| drug& [symbol] =non- | | | | | | | | |
| ionic+surfactant+ | | | | | | | | |
| drug | | | | | | | | |
| — | Non-ionic | (Polyoxyethylene-(20)- | 14.9 | | | | yes | likely |
| | | sorbitanmonoisostearate) - | | | | | | |
| | | Tween 120 | | | | | | |
| | Phospholipid | Phosal 50 PG | Combinations? | | | | | likely |
| | | (Tween80 + PG + PC + | | | | | | |
| | | EtOH) | | | | | | |
| | Phospholipid | Phosal 53 MCT | Combinations? | | | | | likely |
| | Phospholipid | Phosal 50 SA+ | Combinations? | | | | | likely |
| | Phospholipid | Phosal 75 SA | Combinations? | | | | | likely |
| | Phospholipid | PEGylated Phospholipids, | | | | | yes | Very |
| | | e.g. Lipoid MPEG | | | | | | likely |
| | | 5000-DPPE | | | | | | |
| https://www. | Phospholipid | Lecithin -> all natural | 8? | | | | No | yes |
| theherb [symbol] .com/ | | phospholipids | | | | | | |
| files/resource- | | (Lipoid E, S, H, P etc) | | | | | | |
| center/formulation/ | | | | | | | | |
| Emulsifiers_HLB_ | | | | | | | | |
| Values.pdf | | | | | | | | |
| — | Phospholipid | All Lipoid PLs mixed | | | | | No | yes |
| | | with bile salts furhter | | | | | | |
| | | below | | | | | | |
| | Phospholipid | Phosphatidylcholines, | | NA | | | No | yes |
| | | e.g. POPC = 1-Palmitoyl- | | | | | | |
| | | 2-oleoyl-sn-glycero-3- | | | | | | |
| | | phosphocholine, | | | | | | |
| | | CAS-No. 26853-31-6 | | | | | | |
| | Phospholipid | Phosphatidylglycerols, | | NA | | | No | yes |
| | | e.g. DPPG-Na = 1,2- | | | | | | |
| | | Dipalmitoyl-sn-glycero- | | | | | | |
| | | 3-phospho-rac-glycerol, | | | | | | |
| | | sodium salt, | | | | | | |
| | | CAS-No. 67232-81-9 | | | | | | |
| | Phospholipid | Phosphatidylethanolamines, | | NA | | | No | yes |
| | | e.g. DPPE = 1,2- | | | | | | |
| | | Dipalmitoyl-sn-glycero-3- | | | | | | |
| | | phosphoethanolamine, | | | | | | |
| | | CAS-No. 923-61-5 | | | | | | |
| | Phospholipid | Lipoid 16:1/18-1 | | NA | | | No | yes |
| | Phospholipid | Lipoid P LPC 80 | | NA | | | No | yes |
| | Phospholipid | DMPG NA | | NA | | | No | yes |
| | Phospholipid | Lipoid P 75 | | NA | | | No | yes |
| | Phospholipid | Lipoid S 80 | | NA | | | No | yes |
| | Phospholipid | Lipoid E PG/DSPG | | NA | | | No | yes |
| http://www. | Bile salts | sodium taurocholate | 16 | NA | | | No | yes |
| sumobrain.com/ | | (HLB 16) | | | | | | |
| patents/wipo/ | | | | | | | | |
| Nanocapsular- | | | | | | | | |
| formulation-active- | | | | | | | | |
| pharmaceutical- | | | | | | | | |
| ingredients/ | | | | | | | | |
| WO2015189387A1. | | | | | | | | |
| html | | | | | | | | |
| http://www. | Bile salts | sodium taurodeoxycholate | 20.1 | NA | | | No | yes |
| sumobrain.com/ | | (HLB 20.1) | | | | | | |
| patents/wipo/ | | | | | | | | |
| Nanocapsular- | | | | | | | | |
| formulation-active- | | | | | | | | |
| pharmaceutical- | | | | | | | | |
| ingredients/ | | | | | | | | |
| WO2015189387A1. | | | | | | | | |
| html | | | | | | | | |
| | Bile salts | sodium | | NA | | | No | yes |
| | | taurochenodeoxycholate | | | | | | |
| http://www. | Bile salts | sodium cholate | 18 | NA | | | No | yes |
| sumobrain.com/ | | (HLB 18) | | | | | | |
| patents/wipo/ | | | | | | | | |
| Nanocapsular- | | | | | | | | |
| formulation-active- | | | | | | | | |
| pharmaceutical- | | | | | | | | |
| ingredients/ | | | | | | | | |
| WO2015189387A1. | | | | | | | | |
| html | | | | | | | | |
| http://www. | Bile salts | sodium deoxycholate | 16 | NA | | | No | yes |
| sumobrain.com/ | | (HLB 16) | | | | | | |
| patents/wipo/ | | | | | | | | |
| Nanocapsular- | | | | | | | | |
| formulation-active- | | | | | | | | |
| pharmaceutical- | | | | | | | | |
| ingredients/ | | | | | | | | |
| WO2015189387A1. | | | | | | | | |
| html | | | | | | | | |
| http://www. | Bile salts | sodium glycocholate | 16 to 18 | NA | | | No | yes |
| sumobrain.com/ | | (HLB 16-18) | | | | | | |
| patents/wipo/ | | | | | | | | |
| Nanocapsular- | | | | | | | | |
| formulation-active- | | | | | | | | |
| pharmaceutical- | | | | | | | | |
| ingredients/ | | | | | | | | |
| WO2015189387A1. | | | | | | | | |
| html | | | | | | | | |
| NA | | No | yes | | | | | |
| NA | | No | yes | | | | | |
| | Bile salts | Sodium | | NA | | | No | yes |
| | | glycodeoxycholate | | | | | | |
| | Bile salts | sodium | | | | | No | yes |
| | | glycochenodeoxycholate | | | | | | |
| | Non-ionic | Vitamin E TPGS | ca. 13 | | | | Yes | yes |
| | | (d-α-tocopheryl PEG | | | | | | |
| | | 1000 succinate), | | | | | | |
| | | CAS-No. 9002-964 | | | | | | |
| | Non-ionic | Polyoxyl 40 hydrogenated | 14 to 16 | 450 | mg | | Yes | yes |
| | | castor oil (Cremophor or | | | | | | |
| | | Kolliphor RH 40) | | | | | | |
| | Non-ionic | Polyoxyl 35 castor oil | 12 to 14 | 599.4 | mg | | Yes | yes |

| | | | | | | | |
|---|---|---|---|---|---|---|---|
| | | (Cremophor or Kolliphor | | | | | |
| | | EL and ELP) | | | | | |
| | Non-ionic | PEG 15 Hydroxystearate | 14 to 16 | NA | | Yes | Very |
| | | (Solutol HS 15) | | | | | likely |
| | Non-ionic | Polyoxyl 60 hydrogenated | 15 to 17 | NA | | Yes | likely |
| | | castor oil | | | | | |
| | | (Cremophor RH 60) | | | | | |
| | Non-ionic | PEG-25 Hydrogenated | 10.8 | | | Yes | likely |
| | | Castor Oil | | | | | |
| | Non-ionic | Polyoxyl 5 castor oil | 3.7 | | | Yes | |
| | | (PEG- 5 castor oil) | | | | | |
| | | HLB 3.7 | | | | | |
| | Non-ionic | Polyoxyl 9 castor oil | <10.8 | | | Yes | likely |
| | | (PEG- 9 castor oil) | (PEG 25 | | | | |
| | | | Hydrogenated | | | | |
| | | | castor oil) | | | | |
| | Non-ionic | Polyoxyl 15 castor oil | <10.8 | | | Yes | likely |
| | | (PEG-15 castor oil | (PEG 25 | | | | |
| | | | Hydrogenated | | | | |
| | | | castor oil) | | | | |
| Excipient handbook | Non-ionic | Polyoxyl 6 cetostearyl | 10 to 12 | | Topical | Yes | likely |
| | | ether | | | | | |
| Excipient handbook | Non-ionic | Polyoxyl 20 cetostearyl | 15 | | Topical | Yes | likely |
| | | ether | | | | | |
| Excipient handbook | Non-ionic | Polyoxyl 25 cetostearyl | 15 to17 | | Topical | Yes | likely |
| | | ether | | | | | |
| Excipient handbook | Non-ionic | Polyoxyl 9 Lauryl ether, | 13.6 | | Topical | Yes | likely |
| Excipient handbook | Non-ionic | Polyoxyl 10 oleyl ether, | 12.4 | | Topical | Yes | likely |
| | | Brij 96 | | | | | |
| Excipient handbook | Non-ionic | Polyoxyl 20 oleyl ether, | 15.9 | | Topical | Yes | likely |
| | | Brij 98 | | | | | |
| Excipient handbook | Non-ionic | Polyoxyl 21 steryl ether, | 15.5 | | Topical | Yes | likely |
| | | Brij 721 | | | | | |
| Excipient handbook | Non-ionic | Polyoxyl 100 steryl ether | 18.8 | | Topical | Yes | likely |

[symbol]

indicates data missing or illegible when filed

In one embodiment the at least one surfactant is an ionic surfactant.

In one embodiment the at least one surfactant is an anionic surfactant.

For example, bile salts are anionic surfactants. Commonly and preferred used bile salts are described in the earlier section of this application.

In one embodiment the at least one surfactant is a nonionic surfactant.

In one embodiment the at least one surfactant is vitamin E TPGS.

In one embodiment the at least one surfactant is a polyoxyethylene castor oil derivative, which includes but not limited to polyoxyl 5 castor oil (PEG-5 castor oil), polyoxyl 9 castor oil (PEG-9 castor oil), polyoxyl 15 castor oil (PEG-15 castor oil), polyoxyl 35 castor oil (Cremophor EL, or PEG-35 castor oil), polyoxyl 40 hydrogenated castor oil (Cremophor RH 40 or PEG-40 hydrogenated castor oil), Polyoxyl 60 hydrogenated castor oil (Cremophor RH 60 or PEG-60 hydrogenated castor oil).

In one embodiment the at least one surfactant is a polyoxyethylene alkyl ether, which includes but not limited to polyethylene glycol monoacetyl ether, polyethylene glycol monolauryl ether, polyethylene glycol monooleyl ether, polyethylene glycol monostearyl ether.

In one embodiment polyoxyethylene alkyl ether is selected from a group consisting of Polyoxyl 20 cetostearyl ether, Polyoxyl 10 cetyl ether, Polyoxyl 20 cetyl ether, Polyoxyl 23 laur lauryl ether Polyoxyl 23 lauryl ether Polyoxyl 23 lauryl ether, Polyoxyl 10 oleyl ether, Polyoxyl 20 oleyl ether, Polyoxyl 10 stearyl ether and Polyoxyl 21 stearyl ether.

In one embodiment the at least one surfactant is a PEG stearate, e.g. PEG 15 Hydroxystearate (Solutol HS 15, polyethylene glycol (PEG)-15-hydroxystearate) or PEG 32 stearate (Gelucire 48/16, Polyethylene glycol monostearate, Polyoxyl stearate).

In one embodiment the at least one surfactant is a polyoxyethylene sorbitan fatty acid ester, which includes but not limited to Tween 80 (Polysorbate 80, Polyoxyethylene (20) sorbitan monooleate).

In one embodiment the at least one surfactant is selected from the list consisting of all the surfactants labelled as likely or very likely in Table 3.

In one embodiment the at least one surfactant is selected from the list consisting of Vitamin E TPGS, PEG 40 hydrogenated castor oil (Cremophor RH 40) orKolliphor RH40), PEG 32 monostearate (Gelucire 48/16), Gelucire 44/14, Gelucire 50/13, labrasol, and PEG 35 castor oil (Cremophor EL), PEG 15 hydroxystearate (Solutol HS 15), Tween 80 or a mixture of any 3 or any 2 of the surfactants from the list.

Derived from hydrogenated castor oil and ethylene oxide, Kolliphor® RH40 is used as a non-ionic oil-in-water solubilizer and emulsifying agent. BASF catalog contains product information of Kolliphor® RH40.

Labrasol® (Synonym: CAPRYLOCAPROYL MACROGOL-8/POLYOXYL-8 GLYCERIDES Caprylocaproyl polyoxylglycerides PEG-8 Caprylic/Capric Glycerides (FDA IIG)) is a non-ionic water dispersible surfactant composed of well-characterised polyethylene glycol (PEG) esters, a small glyceride fraction and free PEG. Self-emulsify forming a fine dispersion (SMEDDS). Ferromet catalog contains product information of Labrasol®.

Gelucire® 48/16 is a polyethylene glycol monostearate (type I) NF and consists of PEG-32 (MW 1500) esters of palmitic (C16) and stearic (C18) acids.

In one embodiment one of the at least one surfactant is Kolliphor RH40.

In one embodiment one of the at least one surfactant is Gelucire® 48/16.

In one embodiment the pharmaceutical composition of the invention comprises eltrombopag or a pharmaceutically acceptable salt thereof and at least one surfactant, wherein the weight of eltrombopag, based on eltrombopag free acid, is not more than 80%, not more than 60%, suitably not more than 40%, suitably not more than 30%, suitably not more than 25%, suitably not more than 20% of the total weight of eltrombopag and the at least one surfactant. In one embodiment the weight of eltrombopag is not more than 30% of the total weight of eltrombopag and the at least one surfactant. For the sake of clarity, if there are more than one surfactant in the composition, the weight of the at least one surfactant is the total weight of all the surfactants. The weight of eltrombopag is based on the weight of eltrombopag free acid.

In one embodiment the weight of eltrombopag is at least 2%, suitably at least 5%, suitably at least 10% of the total weight of eltrombopag and the at least one surfactant. In one embodiment the weight of eltrombopag is at least 5% of the total weight of eltrombopag and the at least one surfactant.

In one embodiment the pharmaceutical composition of the invention comprises eltrombopag or a pharmaceutically acceptable salt thereof and at least one surfactant, wherein the weight of eltrombopag is from 2% to 50%, suitably from 5% to 40%, suitably from 5% to 30%, suitably from 5% to 25%, suitably from 10 to 20%, of the total weight of eltrombopag and the at least one surfactant. In one embodiment the composition, the weight of eltrombopag is from 10% to 20% of the total weight of eltrombopag and the at least one surfactant.

In one embodiment the pharmaceutical composition further comprises at least one more pharmaceutically acceptable excipients.

In one embodiment the at least one more pharmaceutically acceptable excipients include an anti-oxidant. In one embodiment the weight of the anti-oxidant is not more than 10%, suitably not more than 7%, suitably not more than 5%, suitably not more than 3%, suitably not more than 1%, of the total weight of the pharmaceutical composition. In one embodiment the anti-oxidant is selected from a list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture thereof, suitably 2 of the anti-oxidants from the list. In one embodiment the anti-oxidant is vitamin E. In one embodiment vitamin E is not more than 15%, suitably not more than 10%, suitably not more than 7%, suitably not more than 5% of the total weight of the pharmaceutical composition. In one embodiment vitamin E is 2-15%, suitably 2-10%, suitably 5% of the total weight of the pharmaceutical composition. In one embodiment the anti-oxidant is BHT. In one embodiment BHT is not more than 3%, suitably not more than 1%, suitably not more than 0.5%, suitably not more than 0.2%, suitably not more than 0.1% of the total weight of the pharmaceutical composition. In one embodiment the anti-oxidant is BHA. In one embodiment BHA is not more 3%, suitably not more than 1%, suitably not more than 0.5%, suitably not more than 0.2% of the total weight of the pharmaceutical composition. In one embodiment the anti-oxidant is Propyl gallate. In one embodiment Propyl gallate is not more than 3%, suitably not more than 1%, suitably not more than 0.5% of the total weight of the pharmaceutical composition. In one embodiment the anti-oxidant is EDTA. In one embodiment EDTA is not more than 10%, not more than 5%, suitably not more than 3%, suitably not more than 2% of the total weight of the pharmaceutical composition. In one embodiment EDTA is 1-5%, suitably 1-3%, suitably 2% of the total weight of the pharmaceutical composition.

In one embodiment the pharmaceutical composition consists essentially of or consists of of eltrombopag or a pharmaceutically acceptable salt thereof and at least one surfactant and at least one anti-oxidant. In one embodiment the pharmaceutical composition consists essentially of or consists of eltrombopag or a pharmaceutically acceptable salt thereof and one surfactant and at least one anti-oxidant. In one embodiment the pharmaceutical composition consists essentially of or consists of eltrombopag or a pharmaceutically acceptable salt thereof and at least one surfactant and one anti-oxidant. In one embodiment the pharmaceutical composition consists essentially of or consists of eltrombopag or a pharmaceutically acceptable salt thereof and one surfactant and one anti-oxidant. In one embodiment the anti-oxidant is not more than 10%, suitably not more than 7%, suitably not more than 5%, of the total weight of the pharmaceutical composition. In one embodiment the anti-oxidant is selected from a list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture of.

In one embodiment the at least one anti-oxidant is EDTA.

In one embodiment the pharmaceutical composition of the present invention, preferably in an oral dosage form, comprises eltrombopag or a pharmaceutically acceptable salt thereof, Kolliphor RH40 and EDTA. In one embodiment the pharmaceutical composition of the present invention, preferably in an oral dosage form, consists of eltrombopag or a pharmaceutically acceptable salt thereof, Kolliphor RH40 and EDTA. In one embodiment the weight of eltrombopag is not more than 30% of the total weight of eltrombopag and Kolliphor RH40. Besides Kolliphor RH40, additional surfactants and/or lipids could be added to the pharmaceutical composition of the invention. Typically one or two additional surfactant could be added. Alternatively or additionally one or two lipid could be added. Typically one additional surfactant could be added. Alternatively or additionally one lipid could be added. In one embodiment the pharmaceutical composition of the invention comprises eltrombopag or a pharmaceutically acceptable salt thereof, vitamin E TPGS and Kolliphor RH40. In one embodiment the pharmaceutical composition of the invention comprises eltrombopag or a pharmaceutically acceptable salt thereof, Kolliphor RH40, Maisine and propylene glycol.

Suitably, the at least one more pharmaceutically acceptable excipients include a diluent (also known as filler or bulking agent) and/or a binder and/or a lubricant and/or a disintegrant. Those skilled in the art will recognize that a given material may provide one or more functions in the tablet formulation, although the material is usually included for a primary function.

Diluents provide bulk, for example, in order to make the tablet a practical size for processing. Diluents may also aid processing, for example, by providing improved physical properties such as flow, compressibility, and tablet hardness. Because of the relatively high percentage of diluent and the amount of direct contact between the diluent and the active compound in the typical pharmaceutical formulation, the interaction of the diluent with the active compound is of particular concern to the formulator. Examples of diluents suitable for general use include: water-soluble fillers and water-insoluble fillers, such as calcium phosphate (e.g., di and tri basic, hydrated or anhydrous), calcium sulfate, calcium carbonate, magnesium carbonate, kaolin, spray dried or anhydrous lactose, cellulose (e.g., microcrystalline cellulose, powdered cellulose), pregelatinized starch, starch, lactitol, mannitol, sorbitol, maltodextrin, powdered sugar, compressible sugar, sucrose, dextrose, and inositol. The diluents that do not contain coordinating metals and diluents that are non-reducing sugars are suitable for tablets of the current invention. Suitable diluents for use in this invention include microcrystalline cellulose, powdered cellulose, pregelatinized starch, starch, lactitol, mannitol, sorbitol, and maltodextrin. Unsuitable diluents include calcium phosphate (e.g., di and tri basic, hydrated or anhydrous), calcium sulfate, calcium carbonate, magnesium carbonate, kaolin, and spray dried or anhydrous lactose. In one embodiment of the present invention, the diluent is composed of one or both of Mannitol and microcrystalline cellulose.

Binders impart cohesive properties to the powdered material. Examples of binders suitable for use in the present invention include: starch (e.g., paste, pregelatinized, mucilage), gelatin, sugars (e.g., sucrose, glucose, dextrose, molasses, lactose, dextrin, xylitol, sorbitol), polymethacrylates, natural and synthetic gums (e.g., acacia, alginic acids and salts thereof such as sodium alginate, gum tragacanth, Irish moss extract, panwar gum, ghatti gum, guar gum, zein), cellulose derivatives [such as carboxymethyl cellulose and salts thereof, methyl cellulose (MC), hydroxypropyl methyl cellulose (HPMC), hydroxypropyl cellulose (HPC), hydroxyethyl cellulose (HEC) and ethyl cellulose (EC)], polyvinylpyrrolidone, Veegum, larch arabogalactan, polyethylene glycol, waxes, water, alcohol, magnesium aluminum silicate, and bentonites. In one embodiment of the present invention, the binder comprises polyvinylpyrrolidone (PVP).

Lubricants are generally used to enhance processing, for example, to prevent adhesion of the formulation material to manufacturing equipment, reduce interparticle friction, improve rate of flow of the formulation, and/or assist ejection of the formulations from the manufacturing equipment. Examples of lubricants suitable for use in the present invention include: talc, stearates (e.g., magnesium stearate, calcium stearate, zinc stearate, palmitostearate), stearic acid, hydrogenated vegetable oils, glyceryl behanate, polyethylene glycol, ethylene oxide polymers (e.g., CARBOWAXes), liquid paraffin, sodium lauryl sulfate, magnesium lauryl sulfate, sodium oleate, sodium stearyl fumarate, DL-leucine, and silica derivatives (e.g., colloidal silicon dioxide, colloidal silica, pyrogenic silica, and hydrated sodium silicoaluminate). In one embodiment of the present invention, the lubricant comprises magnesium stearate.

Disintegrants are employed to facilitate breakup or disintegration of the formulation after administration. Examples of disintegrants suitable for use in the present invention include: starches, celluloses, gums, crosslinked polymers, and effervescent agents, such as corn starch, potato starch, pregelatinized starch, modified corn starch, croscarmellose sodium, crospovidone, sodium starch glycolate, Veegum HV, methyl cellulose, microcrystalline cellulose, cellulose, modified cellulose gum (e.g., Ac-Di-Sol R), agar, bentonite, montmorillonite clay, natural sponge, cation exchange resins, ion exchange resins (e.g., polyacrin potassium), alginic acid and alginates, guar gum, citrus pulp, carboxymethylcellulose and salts thereof such as sodium lauryl sulfate, magnesium aluminum silicate, hydrous aluminum silicate, sodium bicarbonate in admixture with an acidulant such as tartaric acid or citric acid. In one embodiment of the present invention, the disintegrant is sodium starch glycolate.

In the foregoing embodiments, the diluent is suitably a combination of mannitol and microcrystalline cellulose, the non-reducing sugar is suitably mannitol, the binder is suitably polyvinylpyrolidone, the lubricant is suitably magnesium stearate, and the disintegrant is suitably sodium starch glycolate.

Oral dosage form is meant to be taken orally, typically as instructed by the medicine manufacturer. Common oral dosage form includes but not limited to solid dosage form such as tablets, capsules, pellets, lozenges, granules and powders and liquid dosage form such as syrup. In one embodiment the oral dosage form is tablet.

In one embodiment the oral dosage form is to be taken into the mouth directly. In one embodiment the oral dosage form is firstly to be suspended/dissolved/dispersed/mixed prior to oral administration. For example a dispersible tablet is firstly dispersed in sufficient liquid, such water/juice prior to oral administration. In one embodiment oral dosage form is a tablet. In one embodiment the tablet is a dispersible tablet. In one embodiment the tablet is to be taken directly into the mouth. In one embodiment the oral dosage form is granules.

In one embodiment the pharmaceutical composition of the invention is in the form of capsule. In one embodiment the pharmaceutical composition of the invention is in the form of soft capsule. In one embodiment the pharmaceutical composition of the invention is in the form of hard capsule. Capsule can be gelatin or non-gelatin based. An example of non-gelatin based capsule is hypromellose (HPMC) based capsule. In one embodiment the hard capsule is HPMC based capsule.

In one embodiment, the pharmaceutical composition of the invention is semi-solid. The composition is prepared by melting the at least one surfactant and incorporating the compound and filling the molten mass into capsules which upon cooling forms a semi-solid in the capsule.

The oral dosage form, suitable tablets, capsules or granules, suitably capsules, containing the pharmaceutical composition of the present invention typically comprise at least about 5 mg or at least about 10 mg of eltrombopag, suitably at least about 10 mg of eltrombopag per dosage form. The oral dosage form, suitable tablets, capsules or granules, suitably capsules, containing the pharmaceutical formulation of the present invention typically comprise at most about 200 mg, 150 mg, 100 mg or 75 mg, suitably at most about 75 mg of eltrombopag per dosage form. In one embodiment the pharmaceutical composition comprises about 10 mg to about 100 mg of eltrombopag, or about 10 mg to 75 mg, or about 5 mg to 75 mg of eltrombopag per dosage form. Preferred embodiments of such dosage form comprises about 12.5 mg, 25 mg, 50 mg, 75 mg, 100 mg or 125 mg of eltrombopag.

In another preferred embodiment, due to the increased bioavailability of eltrombopag in the pharmaceutical composition of the present invention, the oral dosage form comprises less amount of eltrombopag but is bioequivalent sto the corresponding 12.5 mg, 25 mg, 50 mg, 75 mg, 100 mg or 125 mg of the commercial Promacta doses, respectively. In one embodiment the pharmaceutical composition of the present invention comprises about 9 mg, about 17.5 mg, about 19.5 mg, about 35 mg, about 39 mg, about 52.5 mg and about 58.5 mg of eltrombopag, based on the weight of eltrombopag free acid.

In one embodiment the pharmaceutical composition of the present invention, preferably in oral dosage form, suitably tablets, capsules or granules, suitably capsules, is substantially free of, preferably free of, coordinating metals and/or that is substantially free of reducing sugars.

By the term “coordinating metal” and “coordinating metals” and derivatives thereof, as used herein is meant a metal or a metal containing excipient, suitably a diluent, or metal containing tablet coating material, which forms a complex, such as a chelate complex, in the presence of eltrombopag olamine. Examples of such metals include: aluminum, calcium, copper, cobalt, gold, iron, magnesium, manganese and zinc.

By the term “reducing sugar” as used herein is meant a sugar or sugar containing excipient, suitably a diluent, which reacts with eltrombopag or a pharmaceutical acceptable salt thereof, suitably eltrombopag olamine, to form a Maillard product when admixed together. Examples of such reducing sugars include: lactose, maltose, glucose, arabinose and fructose.

In one aspect the invention relates to a process for making the pharmaceutical composition of the invention. For example, homogenization, extrusion, spray granulation, spray layering, spray congealing can be employed.

In one embodiment, the present invention relates to the process for preparing the pharmaceutical composition comprising the steps of homogenizing eltrombopag or a pharmaceutically acceptable salt thereof and the at least one surfactant.

In one embodiment the present invention relates to the process A for preparing the pharmaceutical composition comprising the steps of: a) Melting the at least one surfactant, preferably by heating, preferably by heating above its melting temperature;
b) Adding eltrombopag or a pharmaceutically acceptable salt thereof to the molten mass;
c) Homogenizing the mixture of b); and
d) Formulating mixture c) into a pharmaceutical dosage form, preferably in oral dosage form.

In one embodiment the present invention relates to the process B for preparing the pharmaceutical composition comprising the steps of:
a) Adding eltrombopag or a pharmaceutically acceptable salt thereof to the at least one surfactant;
b) Heating the mixture a) to melt; preferably heating to the temperature above the melting temperature of the at least one surfactant;
c) Homogenizing the mixture of b); and
d) Formulating mixture c) into a pharmaceutical dosage form, preferably in oral dosage form.

In one embodiment the process of A or B comprises a step of adding at least one more pharmaceutically acceptable excipients before or after any one of the steps of a) to c). In one embodiment the step of adding at least one more pharmaceutically acceptable excipients is performed after step c). The at least one more excipients includes, but not limited to one or more of fillers, binders, disintegrants and/or anti-oxidant. Preferably the mixture of c) is mixed with additional filler, binder, disintegrant, lubricant and/or anti-oxidant before formulating into a pharmaceutical dosage form, preferably in oral dosage form, preferably in tablet or in capsule.

In one embodiment the step of adding at least one more pharmaceutically acceptable excipients is performed in Process A. Typically the at least one more pharmaceutically acceptable excipients is added before step b), namely before eltrombopag is added into the molten mass. Typically the at least one more pharmaceutically acceptable excipients includes an anti-oxidant, wherein said anti-oxidant is EDTA. Typically EDTA is dispersed into the molten mass.

Suitably oxidative stress should be avoided during the manufacturing process.

In one embodiment the present invention relates to the process μl for preparing the pharmaceutical composition comprising the steps of:
a) Melting vitamin E TPGS, preferably by heating, preferably by heating above its melting temperature;
b) Optionally adding an anti-oxidant, e.g. EDTA to the molten mass and mixing throughly;
c) Adding eltrombopag or a pharmaceutically acceptable salt thereof to the molten mass;
d) Homogenizing the mixture of c);
e) Formulating mixture d) into a capsule, suitably a HPMC capsule; and
f) Optionally seal the capsule by banding.

In one embodiment, the oral dosage form is a soft or hard gel capsule. The capsules may be prepared according to methods known in the art, suitably filling the pharmaceutical composition of the present invention into the soft or hard capsule, e.g. a standard two-piece hard gelatin capsule. Suitable capsules contain the pharmaceutical composition of the invention in liquid, semi-solid or granules form.

Suitably the pharmaceutical composition of the present invention is in the form of hard capsule. Suitably the shell of the hard capsule is hypromellose (HPMC) based. Suitably the body of the cap of the shell is sealed together by banding. Banding capsule can be performed by applying a small amount of a water/ethanol mixture at the cap and body interface followed by a gentle warming to fuse the two capsule parts together or by capsule banding process where a thin layer of gelatin or HPMC is placed over the edge of the capsule cap and body. Banding provides additional advantage by delaying contact of the drug with gastric fluid.

In one aspect it provides the pharmaceutical composition of the invention for use in the treatment of thrombocytopenia, especially chronic idiopathic thrombocytopenic purpura, aplastic anemia, and acute radiation syndrome (ARS).

In one embodiment the pharmaceutical composition of the invention is for use in the treatment of thrombocytopenia in adult and pediatric patients 1 year and older with chronic immune thrombocytopenia (ITP) who have had an insufficient response to corticosteroids, immunoglobulins, or splenectomy.

In one embodiment the pharmaceutical composition of the invention is for use in combination with standard immunosuppressive therapy for treatment in the first-line treatment of adult and pediatric patients 2 years and older with severe aplastic anemia.

In one embodiment the pharmaceutical composition of the invention is for use in the treatment of patients with severe aplastic anemia who have had an insufficient response to immunosuppressive therapy.

In one aspect it provides a method of treating thrombocytopenia comprising administering to a subject in need thereof a therapeutically effective amount of eltrombopag or a pharmaceutically acceptable salt thereof contained in the pharmaceutical composition of the present invention.

In one aspect it provides a method of treating earlier thrombocytopenia, especially thrombocytopenia naive subject or subject has not received Corticosteroids treatment, comprising administering to a subject in need thereof a therapeutically effective amount of eltrombopag or a pharmaceutically acceptable salt thereof contained in the pharmaceutical composition of the present invention. In one embodiment the pharmaceutical composition of the present invention is used for as first line treatment of thrombocytopenia.

In one aspect it provides a method of treating chemotherapy induced thrombocytopenia (CIT) comprising administering to a subject in need thereof a therapeutically effective amount of eltrombopag or a pharmaceutically acceptable salt thereof contained in the pharmaceutical composition of the present invention.

In one aspect it provides a method of treating low-risk MDS comprising administering to a subject in need thereof a therapeutically effective amount of eltrombopag or a pharmaceutically acceptable salt thereof contained in the pharmaceutical composition of the present invention.

The term “therapeuticallyeffective amount” and derivatives thereof, means that amount of a drug or active ingredient that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In one aspect the present invention provides a method comprising the steps of

a) Preparing a medium comprising a buffering system, a bile salt and phospholipids, wherein the resulting pH is about 6 to 8, about 6.5 to 7.5, preferably about 6.8±0.2, preferably about 6.8;

b) Adding excessive amount of coordinating metals; preferably coordinating metal is calcium, Aluminium or magnesium, preferably coordinating metal is calcium;

c) Optionally waiting for the excessive amount of coordinating metals to completely dissolve in the medium or to saturate in the medium;

d) adding the drug, preferably formulated in a formulation, preferably in a dosage form, into the medium; preferably said drug is eltrombopag, preferably said formulation is the pharmaceutical formulation of the invention typically comprising phospholipids or comprising at least one surfactant, preferably said dosage form is capsule or tablet;

e) Periodically taking solution out in the amount that is sufficient for measuring the dissolved drug concentration; preferably periodically refers to every 15 minutes, preferably every 15 minutes for at least the first hour, preferably after the addition of the drug;

f) Measuring the drug concentration, preferably by UV or by HPLC.

Suitable buffering system has the great capacity of keeping the pH in the range of 6 to 8, about 6.5 to 7.5, preferably about 6.8. Examples of such buffering system are MOPS, HEPES and Meleate buffer. Suitable buffer should not interact itself with calcium so that calcium is kept available. Preferred buffering system is MOPS (3-morpholinopropane-1-sulfonic acid).

Commonly used bile salts are disclosed in this application. Preferred bile salt is sodium taurocholate or sodium glycocholate.

Commonly used phospholipids are diacyl-phospholipids. Preferred is Lecithin.

The above method is useful, for example, in measuring drug dissolution rate in the presence or absence of excessive amount of coordinating metals.

Alternatively for a quick screening of surfactant candidate suitable for influencing the effect of coordinating metals, the above method can be modified in which the surfactant to be tested can be included in the medium of step a) or added prior to, simultaneously or post to the addition of the coordinating metals to the medium, typically prior to the addition of the coordinating metals. In this case the surfactant to be tested is normally not co-formulated with the drug.

In one embodiment the method of measuring drug dissolution rate in the presence of excessive amount of coordinating metals comprising the steps of

a) Prepering a medium comprising 3-morpholinopropane-1-sulfonic acid and Sodium taurocholate+Lecithin in the ratio that the resulting pH is 6.8±0.3; preferably 6.8±0.2, preferably 6.8±0.1, preferably 6.8;

b) Adding excessive amount of coordinating metals; preferably coordinating metal is calcium or magnesium, preferably coordinating metal is calcium;

c) Optionally waiting for the excessive amount of coordinating metals to completely dissolve in the medium or to saturate in the medium;

d) adding the drug, preferably formulated in a formulation, preferably in a dosage form, into the medium; preferably said drug is eltrombopag, preferably said formulation if the pharmaceutical formulation of the invention, preferably said dosage form is capsule or tablet;

e) Periodically taking solution out in the amount that is sufficient for measuring the dissolved drug concentration; preferably periodically refers to every 15 minutes, preferably every 15 minutes for at least the first hour, preferably after the addition of the drug;

d) Measuring the drug concentration.

In one alternative embodiment the medium of step a) is FaSSIF. In another alternative embodiment the medium of step a) is FeSSIF. Both media are commercially available.

The pharmaceutical composition of the invention is capable of mitigating the food effect on eltrombopag, i.e. mitigating the reduction of bioavailability in the presence of food, especially calcium-rich food.

In one embodiment the pharmaceutical composition of the invention, preferably in an oral dosage form, comprising eltrombopag or a pharmaceutically acceptable salt thereof and at least one surfactant, wherein more than 40%, more than 50%, preferably more than 55%, more preferably more than 60%, more preferably more than 65%, more preferably more than 70%, more preferably more than 80%, more preferably more than 90% of eltrombopag is released in a dissolution test performed in the presence of excessive amount of calcium, typically measured at a definite time point after the addition of the drug. Typically the dissolution test is carried out as exemplified in Example 4. Typically the dissolution is measured at least 20 minutes, at least 30 minutes, at least 45 minutes, at least 60 minutes, or at the time point when the dissolution reaches plateau, after the addition of eltrombopag into the test solution. A skilled person would understand when the dissolution reaches plateau, which is typically when there is no more than 5%, no more than 2% drug release over a period of at least 10 minutes or at least 20 minutes.

Calcium forms 1:1 complex with eltrombopag. Thus the term “excessive amount of calcium” refers to the molar ratio of calcium over eltrombopag is higher than 1, suitably higher than 5, suitably higher than 10, suitably higher than 20, suitably higher than 40, suitably higher than 50, suitably higher than 60. Suitably the term “excessive amount of calcium” refers to the molar ratio of calcium over eltrombopag is higher than 40, suitably between 40 to 60, suitably between 40 to 50.

In one embodiment more than 60% of eltrombopag is released from the pharmaceutical composition of the invention in a dissolution test performed in the presence of excessive amount of calcium. In one embodiment the above defined percentage of release is measured at 30 minutes from the addition of eltrombopag into the solution. Suitable the dissolution test is carried out substantially following the dissolution text described in Example 4. In one embodiment more than 60%, preferably more than 70% of eltrombopag is released in a dissolution test performed in the presence of excessive amount of calcium, wherein the molar ratio of calcium over eltrombopag is higher than 40, suitably when the molar ratio of the calcium over eltrombopag is 40 to 50. In one embodiment more than 50%, preferably more than 60% of eltrombopag is released in a dissolution test performed in the presence of excessive amount of calcium, wherein the molar ratio of calcium over eltrombopag is higher than 50, suitably when the molar ration of the calcium over eltrombopag is between 50 to 60. In one embodiment more than 70%, preferably more than 80%, preferably more than 90% of eltrombopag is released in a dissolution test performed in the presence of excessive amount of calcium, wherein the molar ratio of calcium over eltrombopag is higher than 7.5, suitably when the molar ratio of the calcium over eltrombopag is 10 to 20, preferably 15. In one embodiment the dissolution is measured at least 30 minutes, suitably at 30 minutes after the addition of eltrombopag into the test solution. In one embodiment the dissolution is measured at least 30 minutes, suitably at 40 minutes after the addition of eltrombopag into the test solution.

In one embodiment, the amount of eltrombopag released from the pharmaceutical composition of the invention is not reduced by more than 50%, suitably not reduced by more than 40%, suitably not reduced by more than 30%, suitably not reduced by more than 25%, suitably not reduced by more than 20% in the dissolution test carried out in the presence of excessive calcium when compared to that in the absence of calcium, while other conditions are kept identical, typically measured at a definite time point after the addition of the drug. Suitably the dissolution tests are carried out substantially according to Example 4, or according to Example 4. Suitably the amount of released eltrombopag is measured at 40 minutes after the addition of the pharmaceutical composition of the invention to the test medium. Suitably the molar ratio of calcium over eltrombopag is higher than 40, suitably between 40 to 60, suitably between 40 to 50, or suitably 40.

In one embodiment the pharmaceutical composition of the invention, preferably in an oral dosage form, comprising eltrombopag or a pharmaceutically acceptable salt thereof and at least one surfactant, e.g. vitamin E TPGS, wherein plasma eltrombopag AUCO-∞ is not reduced by more than 40%, preferably not reduced by more than 35%, 30%, 25%, 20%, 15%, 10% when the pharmaceutical composition is taken with a high-calcium, moderate-fat, moderate-calorie meal. A standard high-calcium, moderate-fat, moderate-calorie meal contains about 372 calories 20%, about 9 g 10% fat, and about 448 mg 10% calcium. Preferably a standard high-calcium, moderate-fat, moderate-calorie meal contains about 372 calories, about 9 g fat, and about 448 mg calcium. In one preferred embodiment plasma eltrombopag AUCO-∞ is not reduced by more than 20% when the pharmaceutical composition is taken with a high-calcium, moderate-fat, moderate-calorie meal.

In one embodiment the pharmaceutical composition of the invention, preferably in an oral dosage form, comprising eltrombopag or a pharmaceutically acceptable salt thereof and at least one surfactant, e.g. vitamin E TPGS, wherein plasma eltrombopag Cmax is not reduced by more than 40%, preferably not reduced by more than 35%, 30%, 25%, 20%, 15%, 10% when the pharmaceutical composition is taken with a high-calcium, moderate-fat, moderate-calorie meal. In one preferred embodiment plasma eltrombopag Cmax is not reduced by more than 20% when the pharmaceutical composition is taken with a high-calcium, moderate-fat, moderate-calorie meal.

In one embodiment the pharmaceutical composition of the invention, preferably in an oral dosage form, comprising eltrombopag or a pharmaceutically acceptable salt thereof and at least one surfactant, e.g. vitamin E TPGS, wherein plasma eltrombopag AUCO-∞ taken with a high-calcium, moderate-fat, moderate-calorie meal is within about 80% and about 125%, suitably within about 80% and about 100%, suitable within about 80% and about 90% of the AUCO-∞ taken without a meal, e.g. on an empty stomach. In one embodiment the pharmaceutical composition of the invention, preferably in an oral dosage form, comprising eltrombopag or a pharmaceutically acceptable salt thereof and at least one surfactant, e.g. vitamin E TPGS, wherein plasma eltrombopag Cmax taken with a high-calcium, moderate-fat, moderate-calorie meal is within about 80% and about 125%, suitably within about 80% and about 100%, suitable within about 80% and about 90% of the Cmax taken without a meal, e.g. on an empty stomach.

As used herein, the term “about” in relation to a numerical value x means, for example, +/−10%, suitably +/−5%, +/−2%.

In one embodiment there is no requirement to take the pharmaceutical composition “on an empty stomach (1 hour before or 2 hours after a meal)” and/or “at least 2 hours before or 4 hours after other medications (e.g., antacids), calcium-rich foods and/or” in the drug label of the medicine containing the pharmaceutical composition of the invention.

Without further elaboration, it is believed that one skilled in the art can, using the preceding description, utilize the present invention to its fullest extent. The following Examples, therefore, are to be construed as merely illustrative and not a limitation of the scope of the present invention.

### EXAMPLES

### Example 1

### Capsules Comprising Different Amount of Vit E TPGS

Capsules comprising eltrombopag olamine and Vitamin E TPGS shown in Table 3 were prepared.

TABLE 3

| Capsule | Composition | | | |
|---|---|---|---|---|
| strength (mg) | (formulation 1) | mg/capsule | mg/capsule | % w/w |
| 75 | ETB115 | 95.6* | 1.912 | 20.0 |
| | Vit E TPGS | 382 | 7.640 | 80.0 |
| | Total | 477.6 | 9.552 | 100.0 |

*Free acid equivalent

TABLE 4

| | mg/capsule | | mg/capsule | | mg/capsule | |
|---|---|---|---|---|---|---|
| Composition | (formulation 2) | % w/w* | (formulation 3) | % w/w | (formulation 4) | % w/w |
| ETB115 | 95.6* | 20.0 | 95.6* | 24.2 | 63.8 | 17.5 |
| Vit E TPGS | 382 | 80.0 | 300 | 75.8 | 300 | 82.5 |
| Total | 477.6 | 100.0 | 395.6 | 100.0 | 363.8 | 100.0 |
| strength | 75 | | 75 | | 50 | |
| (mg) | | | | | | |

*Free acid equivalent
*As used in the tables of the Examples, % w/w indicates each components' weight percentage of the total composition. For example ETB115 95.6 mg is the 20% of eltrombopag bis-olamine of a total of 477.6 mg of the composition (drug load).

Hard gel capsules were prepared as follows: VitE TPGS is first melted at 60-70 C in a suitable container depending on the batch weight. Then compound is added and continuously mixed with a homogenizer. Aliquots equivalent to the fill weight are filled into the body of the capsules and allowed to cool to room temp. In case of hard capsules, the body is closed with the cap. The body and cap of this two-piece hard shell capsule is securely sealed. The sealing can be done either by spraying

a small amount of a water/ethanol mixture at the cap and body interface followed by a gentle warming to fuse the two capsule parts together or by capsule banding process where a thin layer of gelatin or HPMC is placed over the edge of the capsule cap and body. In both cases, specialized automated machines can be employed.
Capsules containing 50 mg and 25 mg strength were also proportionally prepared according to the same recipe.

TABLE 5

| | | Composition | Composition |
|---|---|---|---|
| | | per unit | per unit |
| | | 7.5 mg | 12.5 mg |
| | | [mg/unit] | [mg/unit] |
| | Composition | (formulation | (formulation |
| Components | per unit [% w/w] | 12) | 13) |
| Eltrombopag | 6.0 | 9.57 | 15.95 |
| Olamine | | | |
| (Vitamin E | 94 | 150.00 | 250.00 |
| TPGS) | | | |
| Hard gelatin | | Size 4 | Size 2 |
| capsule | | | |
| Total | 100.0 | 159.57 | 265.95 |

### Example 2

### Capsules Comprising Eltrombopag, Vit ETPGS and Various Anti-Oxidants

TABLE 6

| | | Composition | Composition | Composition |
|---|---|---|---|---|
| | | per unit | per unit | per unit |
| | | 25 mg | 50 mg | 75 mg |
| | | [mg/unit] | [mg/unit] | [mg/unit] |
| | Composition | (formulation | (formulation | (formulation |
| Components | per unit [%] | 5A) | 5B) | 5C) |
| Eltrombopag | 20.0 | 31.9 | 63.8 | 95.6 |
| Olamine | | | | |
| (Vitamin E | 75 | 119.4 | 238.8 | 358.2 |
| TPGS) | | | | |
| VitE | 5 | 7.9 | 15.9 | 23.8 |
| Hypromellose | — | 1 capsule | 1 capsule | 1 capsule |
| capsule | | | | |
| Total | 100.0 | 159.2 | 318.4 | 477.6 |

TABLE 7

| | | Composition | Composition | Composition |
|---|---|---|---|---|
| | | per unit | per unit | per unit |
| | | 25 mg | 50 mg | 75 mg |
| | | [mg/unit] | [mg/unit] | [mg/unit] |
| | Composition | (formulation | (formulation | (formulation |
| Components | per unit [%] | 6A) | 6B) | 6C) |
| Eltrombopag | 20.0 | 31.9 | 63.8 | 95.6 |
| Olamine | | | | |
| (Vitamin E | 79.9 | 127.22 | 254.44 | 381.67 |
| TPGS) | | | | |
| BHT | 0.07 | 0.11 | 0.22 | 0.33 |
| Hypromellose | — | 1 capsule | 1 capsule | 1 capsule |
| capsule | | | | |
| Total | 100.0 | 159.2 | 318.4 | 477.6 |

TABLE 8

| Capsule | Composition | | |
|---|---|---|---|
| strength (mg) | (formulation 7) | mg/capsule | % w/w |
| 75 | ETB115 | 95.6* | 20.0 |
| | VitE TPGS | 381.45 | 79.9 |
| | BHA | 0.55 | 0.12 |
| | Total | 477.6 | 100.0 |

TABLE 9

| Capsule | Composition | | |
|---|---|---|---|
| strength (mg) | (formulation 8) | mg/capsule | % w/w |
| 75 | ETB115 | 95.6* | 20.0 |
| | VitE TPGS | 380.46 | 79.66 |
| | Propyl Gallate | 1.54 | 0.322 |
| | Total | 477.6 | 100.0 |

*Free acid equivalent

TABLE 10

| | | Composition | Composition | Composition |
|---|---|---|---|---|
| | | per unit | per unit | per unit |
| | | 25 mg | 50 mg | 75 mg |
| | | [mg/unit] | [mg/unit] | [mg/unit] |
| | Composition | (formulation | (formulation | (formulation |
| Components | per unit [%] | 14A) | 14B) | 14C) |
| Eltrombopag | 20.0 | 31.9 | 63.8 | 95.7 |
| Olamine | | | | |
| (Vitamin E | 77.9 | 124.0 | 248.0 | 372.0 |
| TPGS) | | | | |
| Edetate | 2.1 | 3.3 | 6.7 | 10.0 |
| Disodium | | | | |
| Hypromellose | — | 1 capsule | 1 capsule | 1 capsule |
| capsule | | | | |
| Total | 100.0 | 159.2 | 318.5 | 477.7 |

TABLE 11

| | | Composition | Composition | Composition |
|---|---|---|---|---|
| | | per unit | per unit | per unit |
| | | 17.5 mg | 52.5 mg | 58.5 mg |
| | | [mg/unit] | [mg/unit] | [mg/unit] |
| | Composition | (formulation | (formulation | (formulation |
| Components | per unit [%] | 14D) | 14E) | 14F) |
| Eltrombopag | 20.0 | 22.33 | 66.99 | 74.65 |
| Olamine | | | | |
| (Vitamin E | 77.9 | 86.80 | 260.40 | 290.16 |
| TPGS) | | | | |
| Edetate | 2.1 | 2.33 | 7 | 7.8 |
| Disodium | | | | |
| Hypromellose | — | 1 capsule | 1 capsule | 1 capsule |
| capsule | | | | |
| Total | 100.0 | 115.2 | 334.4 | 372.6 |

Capsule formulations containing Eltrombopag, vit ETPGS and an anti-oxidant were manufactured in a similar manner as described in example 1. The anti-oxidant was added after vit ETPGS was melted and it is further mixed by stirring. Drug was added then to the mixture. Whenever possible, it is preferred to avoid/reduce oxidative stress during the manufacture process and to minimize exposure to water during storage.

### Example 3

### Capsule Formulations Comprising Eltrombopag and Various Surfactants

TABLE 12

| Capsule | Composition | | |
|---|---|---|---|
| strength (mg) | (formulation 9) | mg/capsule | % w/w |
| 75 | ETB115 | 95.6 | 25.8 |
| | Kolliphor Rh 40 | 275.0 | 74.2 |
| | Total | 370.6 | 100.0 |
| 50 | ETB115 | 63.8 | 18.8 |
| | Kolliphor Rh 40 | 275.0 | 81.2 |
| | Total | 338.8 | 100.0 |

TABLE 13

| Capsule | Composition | | |
|---|---|---|---|
| strength (mg) | (formulation 10) | mg/capsule | % w/w |
| 75 | ETB115 | 95.6 | 25.8 |
| | Vit E TPGS | 137.5 | 37.1 |
| | Kolliphor Rh 40 | 137.5 | 37.1 |
| | Total | 370.6 | 100.0 |
| 50 | ETB115 | 63.8 | 18.8 |
| | Vit E TPGS | 137.5 | 40.6 |
| | Kolliphor Rh 40 | 137.5 | 40.6 |
| | Total | 338.8 | 100.0 |

Capsule formulations containing eltrombopag and various surfactants were manufactured in a similar manner as described in example 1.

TABLE 14
(formulation 11)
ETB115-ORA-0024-004 (MEPC 7)

| Composition^{A} | Gm | % w/w |
|---|---|---|
| VitETPGS | 15 | 18.75% |
| Span 80 | 15 | 18.75% |
| Miglyol 812N | 5 | 6.25% |
| Labrasol | 25 | 31.25% |
| Ethanol | 20 | 25% |
| | 80 | 100% |

^{A}Placebo MEPC was prepared first followed by addition of ETB115 at 2% w/w (20 mg/mL)

TABLE 15
(formulation 15)

| MEPC 3 | % |
|---|---|
| Kolliphor RH 40 | 45 |
| Maisine CC | 27 |
| Propylene glycol | 18 |
| Ethanol | 10 |
| Total | 100 |

50 mg ETB115 is suspended in 1 ml of the above preconcentrate. (2×size 0 capsules each with 0.5 ml of the formulation=equivalent to 50 mg ETB115 was tested for dissolution in MOPS+SIF buffer)

Example 4
Dissolution test

| Principle | Measurement of the amount of drug substance | |
|---|---|---|
| | dissolved in a dissolution apparatus 2 (paddle) | |
| | according to Ph. Eur. 2.9.3 “Dissolution for Solid | |
| | Dosage Forms” or USP <711> “Dissolution”. | |
| | Determination by UV detection. | |
| Reagents | | |
| MOPS Buffer | e.g. Sigma Aldrich, AR grade or equivalent | |
| (3- | | |
| morpholinopropane- | | |
| 1-sulfonic acid) | | |
| SIF powder | e.g. Bio-relevant, FaSSIF/FeSSIF/FasssGF | |
| (Sodium | powder or equivalent | |
| taurocholate + | | |
| Lecithin) | | |
| Sodium Hydroxide | AR grade or equivalent | |
| pellets | | |
| Water | Elix water or equivalent | |
| Potassium phosphate | AR grade or equivalent | |
| monobasic (KH₂PO₄) | | |
| Potassium hydroxide | AR grade or equivalent | |
| pellets | | |
| Ortho-phosphoric acid | AR grade or equivalent | |
| (85% or dilute) | | |
| Tween 80 | AR grade or equivalent | |
| (Polyoxyethylene | | |
| sorbitan monooleate) | | |
| Material | | |
| Filter | ROBY25/GF55 glass fiber filter 0.7 μm, or | |
| | Pall Acrodisc PSF GxF/Glass 1 μm | |
| Equipments | | |
| Apparatus | USP apparatus 2 (paddles) | |
| UV spectrophotometer | e.g. PerkinElmer Lambda UV365 or equivalent | |
| Sinkers | CUSBSK-JP | |
| Dissolution Condition | | |
| Test Medium | MOPS + SIF Buffer | |
| | Dissolve 20.9 g of MOPS buffer in 1 liter of | |
| | water and mixed well. Adjust the pH 6.8 ± 0.1 | |
| | with Sodium hydroxide. Add 0.74 g of SIF | |
| | powder and stir gently until mixed well | |
| Test Medium for | 0.5% Tween 80 in MOPS buffer pH 6.8 | |
| Reference Preparation | Dissolve 20.9 g of MOPS buffer in 1 liter of | |
| | water and mixed well. Adjust the pH 6.8 ± 0.1 | |
| | with Sodium hydroxide. Add 5 ml (5.2 g) | |
| | Tween-80 in 1 liter MOPS buffer and Mixed | |
| | Well. | |
| Speed of Rotation | 100 ± 3 rpm | |
| Volume of test | 900 ml | |
| medium | | |
| Temperature | 37.0 ± 0.5° C. | |
| Number of units | Examine the prescribed number of units | |
| tested | according to Acceptance Table 1 of current USP, | |
| | Ph. Eur. or acceptance table 6.10-1 of JP | |
| | (minimum of 6, 1 per vessel) | |
| Test Procedure | | |
| Test solution (profile) | This test can be carried out by an automated | |
| | dissolution test system. In dissolution profiles, | |
| | Samples are required to collect at 30, 45, 60, 90 | |
| | and 105 minutes (Infinity testing at 200 rpm). Do | |
| | not replace the medium removed. At each time | |
| | point, withdraw 10 mL of the solution and | |
| | immediately filter through a Pall Acrodisc PSF | |
| | GxF/Glass 1 μm Automated Certified syringe | |
| | filters (recommended) or ROBY25/GF55 glass | |
| | fiber filter 0.7 μm. Collect the test solution into a | |
| | container for UV measurement. | |
| Calcium challenge | At 30 minutes prior to adding the dosage form, 50 | |
| procedure: | mg or 427 mg or 450 mg of calcium chloride is | |
| | added and allowed to dissolve completely. After | |
| | 30 minutes the dosage form is added and this is | |
| | considered as time 0 for defining the dissolution | |
| | profile | |
| | It should be noted that the above amounts reflect | |
| | 50 mg or 427 mg or 450 mg of elemental calcium, | |
| | respectively. | |
| Note: | Sampling time for profile generation may be | |
| | adapted based on project need. | |
| Reference solutions | For 12.5 mg, 25 mg, 50 mg and 75 mg | |
| | Prepare in duplicate. Weigh approximately 14.0 | |
| | mg (±1.4 mg) of ETB115 reference substance | |
| | into a 200 mL volumetric flask. Fill the flask with | |
| | 40 mL of water and sonicate for a maximum of 5 | |
| | minutes. Fill to 90% with dissolution medium for | |
| | reference preparation with continuous sonication | |
| | and shaking for 30 minutes or until dissolved | |
| | completely. Dilute to volume with dissolution | |
| | medium and mix well. | |
| | This corresponds to 0.055 mg/mL as free acid of | |
| | ETB115. | |
| | This solution is stable for 14 days in ambient | |
| | conditions. | |
| Standard repeatability | The relative standard deviation of the response | |
| | factors of standard 1 is ≤2%. | |
| Standard accuracy | The difference in the mean response factor | |
| | between standard 1 and standard 2 is ≤2%. | |
| Evaluation | Determine the absorbance of the test medium, | |
| | reference and test solutions using a suitable | |
| | spectrophotometer. | |
| Cell (Quartz) | 12.5 mg, 25 mg | 0.2 cm |
| | 50 mg, 75 mg | 0.1 cm |
| Blank | Test medium | |
| Wavelengths | Sample wavelength | 424 nm |

| | |
|---|---|
| Calculations | Calculate the response factor, RF, for each |
| | standard measurement |
| | $RF = \frac{{W}_{s} \times P \times F}{{A}_{s} \times 100}$ |
| Where | |
| Aₛ | Absorbance of standard solution |
| P | Purity of standard (%) |
| Wₛ | Weight of standard (mg) |
| 100 | Correction for percent purity value |
| F | Salt to base conversion factor = MW1/ |
| | MW2 = 0.784 |
| MW1 | Molecular weight of drug substance (as acid) |
| | (442.48 mg/mmole) |
| MW2 | Molecular weight of drug substance (as salt) |
| | (564.67 mg/mmole) |
| | Determine a mean response factor (MRF) from |
| | the standard measurements used in the |
| | quantitation and determine the percent relative |
| | standard deviation (% RSD). |
| | Calculate the quantity of ETB115 released as a |
| | percentage of the label claim, as shown below: |
| | $\% label claim = \frac{{A}_{u} \times MRF \times {V}_{m} \times {DF}_{u} \times 100}{{DF}_{s} \times LC}$ |
| Where | |
| Aᵤ | Absorbance of sample solution |
| MRF | Mean response factor |
| Vₘ | Original media volume |
| DFᵤ | Dilution factor of sample |
| DFₛ | Dilution factor of standard |
| LC | Label claim (mg/Capsule) |
| | When sample aliquots are removed from the |
| | dissolution vessels for analysis, the amounts |
| | removed at each sampling interval are significant |
| | (more than 1 or 2 mL) then mathematical |
| | corrections are needed to compensate for |
| | preceding withdrawals. The following equation |
| | is applied to uncorrected data to correct for both |
| | sample volume removal and media replacement |
| | (if any): |
| | $\begin{matrix}Equation ( 1 ) \\ {C}_{n , corr} = {C}_{n} \times \frac{{V}_{m} - ( {V}_{s} - {V}_{r} ) ( n - 1 )}{{V}_{m}} + \frac{{V}_{s}}{{V}_{m}} {\sum }_{i = 1}^{n - 1} {C}_{i}\end{matrix}$ |
| Where | |
| C_{n,corr} | Corrected ETB115 released (% label claim) at |
| | sample interval n |
| Cₙ | Uncorrected ETB115 released (% label claim) at |
| | sample interval n |
| Vₘ | Original media volume |
| Vₛ | Volume of sample removed |
| Vᵣ | Media replacement volume |
| Cᵢ | Uncorrected ETB115 released % label claim at |
| | previous sample interval i |
| | When the specified media replacement volume is |
| | equal to the sample removal volume Equation (1) |
| | reduces to |
| | ${C}_{n , corr} = {C}_{n} + \frac{{V}_{s}}{{V}_{m}} {\sum }_{i = 1}^{n - 1} {C}_{i}$ |

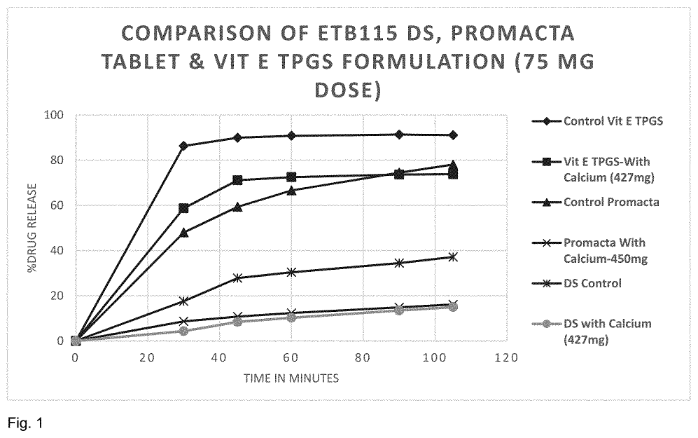

Dissolution test according to EXAMPLE 4 was carried out for formulation 1 in 75 mg in comparison to Promacta. As shown in FIG. 1, vitamin E TPGS has effectively maintained the dissolution rate in the presence of excessive amount of calcium. Similar effect was also observed for formulation 1 in 25 mg and 50 mg strength (data not shown).

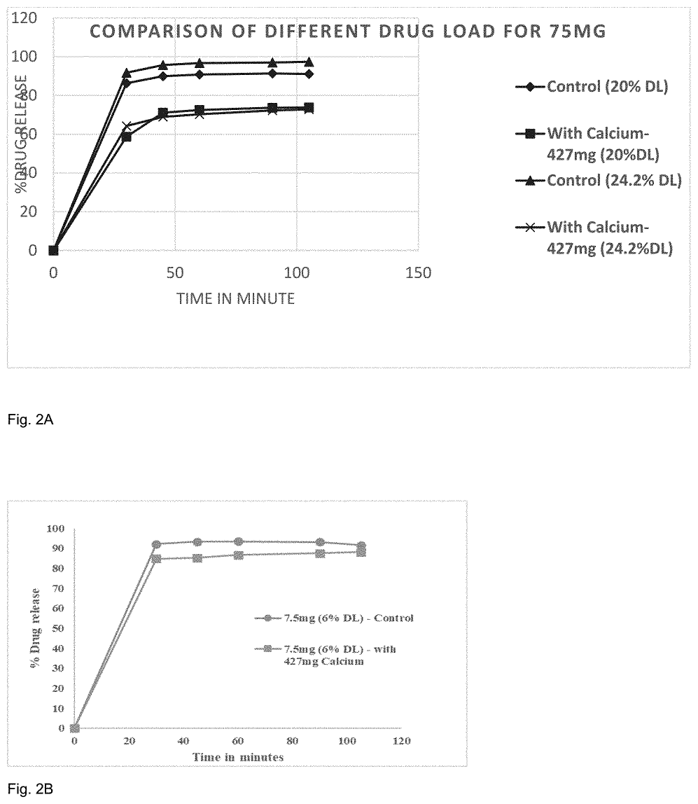

Similar anti-calcium effect was also seen in formulations of different drug loads (FIG. 2A for formulation 3 and FIG. 2B for formulation 12).

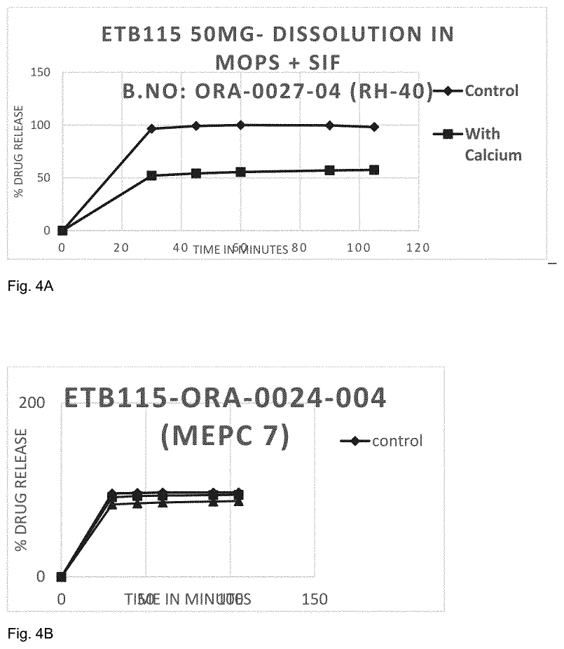

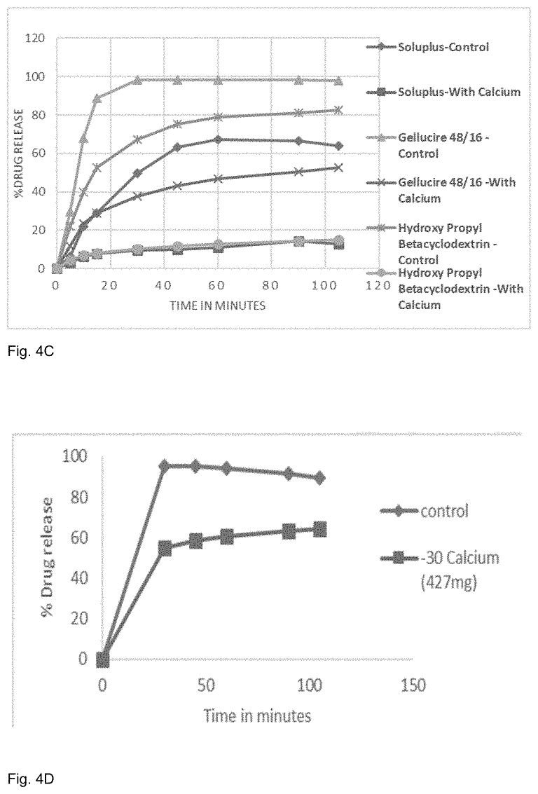

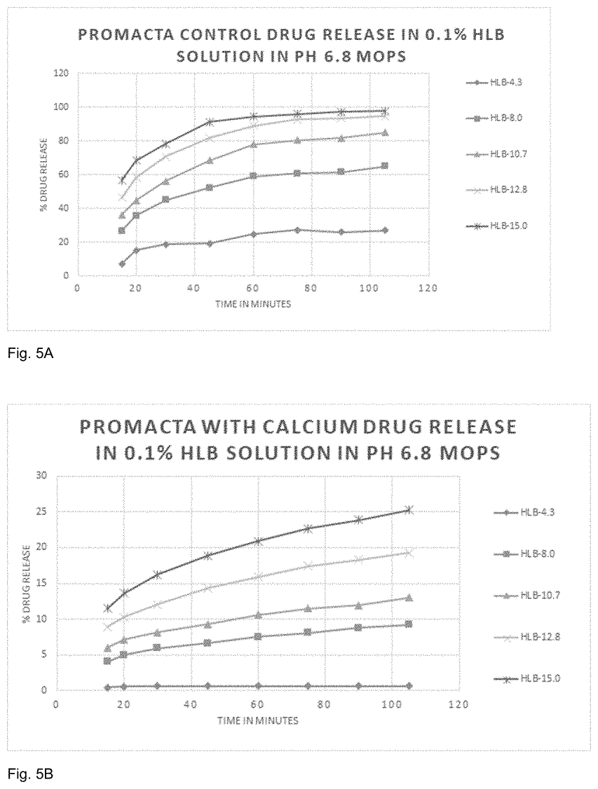

The anti-calcium effect by other surfactants or mixture of surfactants is shown in FIG. 4.

Data in FIG. 4D were generated following EXAMPLE 4

a. 0.5% Gelucire 48/16 (0.5%=4.5 grams) was added to 900 ml MOPS+SIF buffer,

b. after 40 minutes—427 mg Ca (solution) was added to the above media.

c. After 30 minutes—the Promacta 75 mg tablet was added to this media (containing Gelucire 48/16 and Ca) and tested for dissolution.

d. Separate “Control—without the addition of calcium” dissolution was also done for comparison.

Similarly, other suitable surfactants can be selected as above.

### Example 5

### Impact of Vitamin E TPGS Concentration and Sequence of Calcium Addition on Dissolution

To further understand effect of Vitamin E TPGS on drug release, dissolution of 75 mg Promacta® tablets was performed; (1) with different concentrations of Vitamin E TPGS and (2) sequence of calcium addition in MOPS buffer.
Dissolution in MOPS buffer to understand the effect of Vitamin E TPGS on % release from 75 mg Promacta® tablets

TABLE 16

| | | Vitamin E TPGS |
|---|---|---|
| Experiment | Amount of calcium | concentration % w/v, |
| no | (addition time¹) | (addition time¹) |
| 1 | 50 mg elemental | 0.1, (−70 min) |
| 2 | Calcium (−30 min) | 0.3, (−70 min) |
| 3 | | 0.5, (−70 min) |
| 4 | 50 mg elemental | 0.1, (−70 min) |
| 5 | Calcium (+60 min) | 0.3, (−70 min) |
| 6 | | 0.45, (−70 min) |

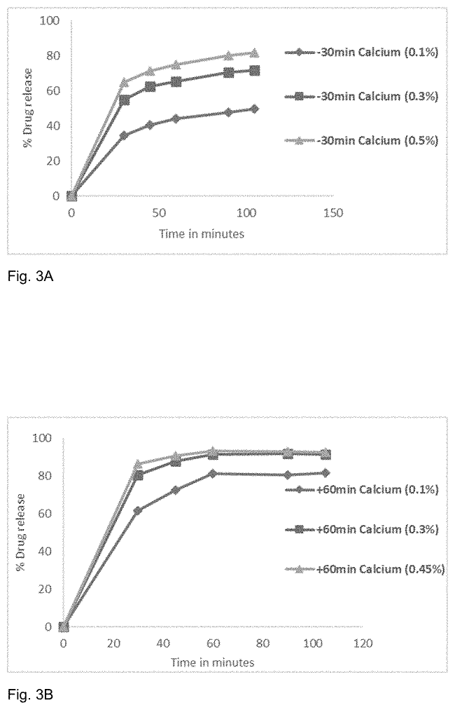

Results of the dissolution from group with calcium added 30 min before addition of Promacta® tablet showed Vitamin E TPGS concentration dependent release (FIG. 3A). As Vitamin E TPGS is increased from 0.1 to 0.5% w/v in dissolution media, the % of drug release at 105 min increases from 50 to 82%.

On the contrary, when the calcium is added 60 min after addition of Promacta® tablets, the release is relatively unaffected by the concentration of Vitamin E TPGS at 0.3 and 0.45% w/v (FIG. 3B).

From above results, the effect of Vitamin E TPGS can be summed up as 1) eltrombopag dissolution shows concentration dependence when calcium is present in the media from the beginning which is likely to be the situation in-vivo, 2) eltrombopag once solubilized in dissolution media in presence of Vitamin E TPGS, effect of calcium mediated drop in dissolution is mitigated. So, partially solubilized suspension of DS in Vitamin E TPGS could contribute to reduce calcium mediated food effect.

### Example 6

### Effects of Surfactants with Different HLB Value on Drug Dissolution

HLB solution preparation:
HLB-8 solution:
Mixed 32.5 ml Span-80 and 17.5 mlTween-80.
HLB-10.7 solution:
Mixed 20 ml Span-80 and 30 mlTween-80.
HLB-12.8 solution:
Mixed 10 ml Span-80 and 40 mlTween-80.
Dissolution media Preparation:
0.1% solution of HLB-4.3 (Span-80) in MOPS:
Mixed 2 ml of SPAN-80 with 2000 ml of MOPS buffer. Mixed well.
0.1% solution of HLB-8 in MOPS:
Mixed 2 ml of HLB-8 with 2000 ml of MOPS buffer. Mixed well.
0.1% solution of HLB-10.7 in MOPS:
Mixed 2 ml of HLB-10.7 solution with 2000 ml of MOPS buffer. Mixed well.
0.1% solution of HLB-12.8 in MOPS:
Mixed 2 ml of HLB-12.8 Solution with 2000 ml of MOPS buffer. Mixed well.
0.1% solution of HLB-15.0 (Tween-80) in MOPS:
Mixed 2 ml of Tween-80 with 2000 ml of MOPS buffer. Mixed well.

### Control Preparation:

Dropped 1 Promacta tablet, kept in sinker in 900 ml with respective dissolution media. After each specified time point, sample was withdrawn automatically and filtered through ROBY 25/GF 55 Filter. Dissolution results are given in FIG. 5A.
With 427 mg calcium Preparation:
Added 1185 mg CaCl₂) 30 minutes before dropping 1 Promacta tablet. Dropped 1 tablet, kept in sinker in 900 ml with respective dissolution media. After each specified time point, sample was withdrawn automatically and filtered through ROBY 25/GF 55 Filter. Dissolution results are given in FIG. 5B.
The results showed that with surfactants with higher HLB value resulted in higher solubilization of ETB115 and had stronger anti-calcium effect.

### Example 7

### Compositions Comprising Phospholipids

Lipid based formulations were prepared with varying ratio of the components as per weights in Table 17. Lipoid E80 S(Cas No. 93685-90-6), glycocholic acid, glycerol and ETB115 were first dissolved in organic solvent in a round bottom flask to obtain a optically clear solution and then solvent was evaporated gradually, which results is solid gel like cake. The resulting solid gel cake readily disperses with water with brief use of sonication and heat. The resulting viscous fluid gel like formulation was filled into hard gelatin capsules by weight and allowed to cool to room temperature. The body is closed with the cap and used for dissolution studies.

Alternatively the resulting solid gel cake can be directly filled into capsules by further extrusion or can be hydrated with required amount of non-aqueous hydrophilic or lipophilic solvent for filling in soft gel capsules.

The lipid particles size tested upon dilution with water were found to be around 190 nm in size with good uniformity as determined by Malvern dynamic light scattering technique. We anticipate that the true particle size of the formulations would be much smaller if tested undiluted.

Surprisingly, we found that lipid: drug weight ratio of 5:1 or 9:1 have almost same degree of drug associated, it was 94% for 5:1 vs 1⁰⁴% for 9:1 ratio signifying almost majority of drug is associated with the lipid or micelle. Association was measured by filtration through 0.2 micron filter and concentration of the filtrate was assessed using Liquid chromatography. The addition of bile salt and glycerol improves the dispersion and hydration time. The increasing amount of bile salt decreases the mixed micelles particle size. Furthermore, upon dilution with GI simulated fluids, these particles were found be stable in both Fed (FeSSIF) and Fasted (FaSSIF) state simulated media as observed by no dramatic change in the particle size. In fact, in presence of Fed state media, the lipid based mixed micelle formulation dispersed more readily into micelle-based formulation. Formulation also showed increased solubility in both fast and fed simulated GI fluids which justifies that compound had less tendency to precipitate upon dilution and a lipid dispersion is formed upon dilution (Table 19). Lastly, the improved solubility of more than 100× upon dilution with bio relevant media translated to enhanced dissolution in dissolution studies (following the protocol as described in EXAMPLE 4) when compared to control Promacta formulation. Mixed micelle formulation L-F2 showed >90% dissolution within 30 min and showed that negative effect of calcium on dissolution is being mitigated by using lipid based mixed micelle formulation.

Lipid Complex with Hydrophillic Co-Solvent (F3 and F4):

To prepare lipid complex, capsules were prepared as per weights in Table 18. Lipoid P LPC (Cas No. 9008-30-4) or Lipoid E80 S, PEG 300 co-solvent and ETB115 were first dissolved in organic solvent (DCM/Methanol 1:1) in a round bottom flask and then solvent was evaporated which results is solid cake. This solid cake is then hydrated with required amount of distilled water which results into a highly viscous gel like formulation. Upon, rehydration the fluid aliquots equivalent to the fill weight and filled into the body of the capsules and allowed to cool to room temperature. In case of hard capsules, the body is closed with the cap.

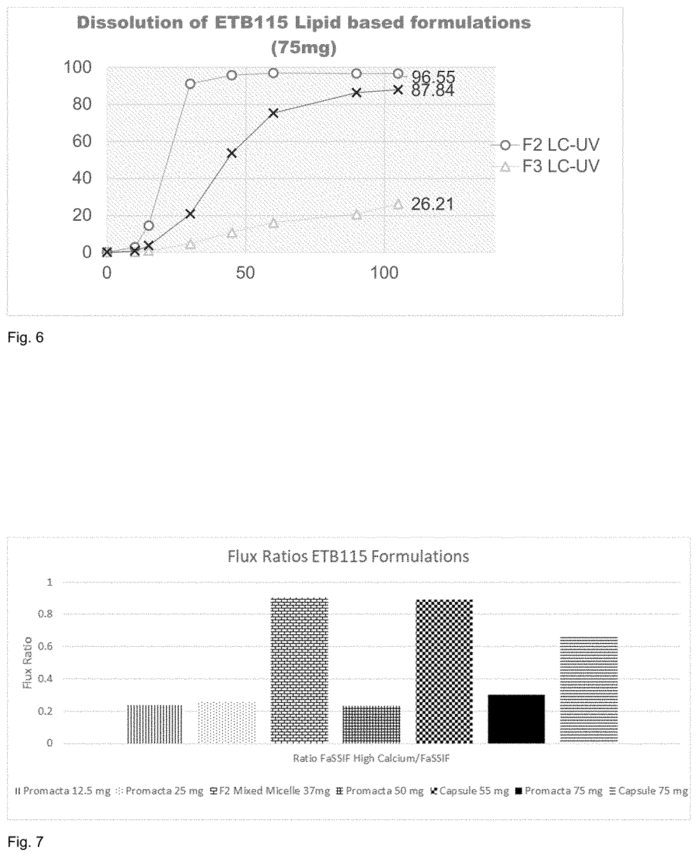

These lipid complex formulation dispersed with water with brief use of sonication and heat. The lipid particles formed after hydration with water are around 31 nm in size (LPC lipid L-F4) and around 490 nm (Lipoid E 80S L-F3) with good uniformity as determined by malvern dynamic light scattering tecnhique. The compoisition L-F4 was selected based on the highest association, stability upon dilution with biorelevant media and also due to the small micelle like particle formation. The wieght ratio of 7:1 was found to provide the highest level of association/entrapment of drug, close to 100%. Alternately, 5:1 ratio can also be utilized as it shows minimum impact on association (L-F4-B) Lipid complex formulation showed enhanced stability upon dilution in biorelevant media as evident by no major change in size (Table 19) and also provided better solubility. The enchanced solubility in biorelevant media also resulted in better dissolution profile (70-80% in 60 min) compared to control Promatca in the dissolution studies described in EXAMPLE 4. The results are shown in FIG. 6. Although, the dissolution of F3 was low, we believe that F3 formulation could be furthur optimized to improve solubility and stability as we had some technical processing challenges while scaling up the formulations.

In summary, it was demonstrated that lipid based formulation, mixed micelle or lipid complexes both can facilitate high association of the compound with lipids which in turn resulted in improved solubilization upon dilution in GI biorelevant media and provided close to complete dissolution of drug in presence of calcium where perhaps compound was shielded from interaction with calcium.

### Examples L-F2

TABLE 17
Capsules comprising Lipid based Micelles
with bile acid are shown below:

| | | mg/capsules | | mg/capsule |
|---|---|---|---|---|
| | | (formulation | | (formulation |
| Composition | % w/w* | L-F2) | % w/w | L-F2-B) |
| ETB115 | 5.5 | 95.6 | 14.2 | 95.6 |
| Lipoid E80 S | 49.6 | 860 | 71.2 | 478.0 |
| Lipoid P LPC 80 | NA | NA | NA | NA |
| Na Glycocholic acid | 38.4 | 666.2 | 12.5 | 83.7 |
| PEG 300 | NA | NA | NA | NA |
| Glycerol | 6.4 | 111.7 | 2.1 | 14.3 |
| Physical apperance | | Wine red, | | Wine red, |
| before hydration | | slightly viscous | | slightly viscous |
| | | gel cake | | gel cake |
| Hydration Water in | | 1000 | | 1195 |
| gms | | | | |
| Total | 100 | 1733.5 | 100 | 671.59 |
| % Association | | 104 | | 94 |
| Lipid:drug ratio wt | | 9 | | 5 |

*Free acid equivalent
*As used in the tables of the Examples, % w/w indicates each components' weight percentage of the total composition. For example ETB115 95.6 mg is the 5.5% of eltrombopag bis-olamine of a total of 1638 mg of the composition (drug load).

### Example L-F4-B and-C L-F3)

TABLE 18
Capsules containing Lipoid P LPC, Lipoid E 80 S Lipid complex
formulations are shown below:

| | | mg/capsule | | mg/capsule | | mg/capsule | | mg/capsule |
|---|---|---|---|---|---|---|---|---|
| | % | (formulation | % | (formulation | % | (formulation | % | (formulation |
| Composition | w/w | L-F4) | w/w | L-F4-B) | w/w | L-F4-C) | w/w | L-F3) |
| ETB115 | 11.4 | 95.6 | 13.8 | 95.6 | 16.7 | 95.6 | 9.1 | 95.6 |
| Lipoid | NA | NA | 69.0 | 478.0 | 83.3 | 478.0 | 82.0 | 860.4 |
| E80 S | | | | | | | | |
| Lipoid P | 79.7 | 669.8 | NA | NA | NA | NA | NA | NA |
| LPC 80 | | | | | | | | |
| Na | NA | | | | | | | |
| Glycocholic | | | | | | | | |
| acid | | | | | | | | |
| PEG 300 | 8.9 | 74.56 | 17.2 | 119.5 | | 0.0 | 8.9 | 93.2 |
| Glycerol | | | | | | | | |
| Physical | | Wine red, | | Wine red, | | Wine red, | | Wine red, |
| apperance | | highly | | highly | | highly | | highly |
| before | | viscous cake | | viscous cake | | viscous cake | | viscous cake |
| hydration | | | | | | | | |
| Hydration | | 1250 | | 1195 | | 1195.0 | | 1623.0 |
| Water in | | | | | | | | |
| gms | | | | | | | | |
| Total | 100 | 839.96 | 100 | 693.1 | 100.0 | 573.6 | 100.0 | 1049.2 |
| % | | 98 | | 100 | | 42.0 | | 101.2 |
| Association | | | | | | | | |
| Lipid:drug | | 7 | | 5 | | 5 | | 9 |
| ratio wt | | | | | | | | |

*Free acid equivalent
* As used in the tables of the Examples, % w/w indicates each components' weight percentage of the total composition. For example ETB115 95.6 mg is the 11.4% of eltrombopag bis-olamine of a total of 744.36 mg of the composition (drug load).

TABLE 19
Particle size and solubility data for L-F2 mixed
micelle and L-F4 lipid complex formulations

| | | | Fraction |
|---|---|---|---|
| | Dilution | Particle size | solubility |
| Formulation Type | Media | (nm) | (mg/ml) |
| Formulation L-F2 | Water | 186.7 | na |
| mixed micelle | FaSSIF | 241.4 | 5.35 |
| | FeSSIF | 46.14 | 6.41 |
| Formulation L-F4 lipid | Water | 31.07 | na |
| complex | FaSSIF | 22.11 | 4.59 |
| | FeSSIF | 25.89 | 4.88 |

### Example 8

### Pampa Test

A study combining the experimental determination of dissolution and simultaneous flux through an artificial lipidic membrane was conducted on a 12.5, 25, 50 and 75 mg dose of Promacta and a 55 and 75 mg dose of formulation 1 (in Capsule) of the invention. In addition, a mixed micelle formulation (F2) was evaluated at a 37 mg dose.

FaSSIF media was prepared according to instruction from biorelevant.com. Additionally, each media are prepared containing 450 mg of elemental calcium, to mimic a high calcium meal and understand the impact of calcium and dissolution and resultant flux. This was achieved by adding calcium chloride to the media. The media are added, 900 mL in total, to a USP II apparatus, equipped with a paddle attachment for stirring. The dosage unit was introduced to each media. Dissolution analysis were conducted in each media: FaSSIF, V2; FaSSIF, V2+Calcium. This described set-up is considered the donor compartment.

A receiver compartment was introduced into the USP II dissolution set-up. This consists of a minaturized USP II paddle set-up. The bottom of the vessel has a 0.45 um PVDF membrane. Concentrations of eltrombopag in the donor vessel and receiver vessel are monitored with fiber optic probes.

The capsule formulation containing about 80% vitamin E TPGS, as well as the F2 formulation has improved the Fasted+High Calcium/Fasted flux ratio significantly: Ratio of 0.9 for the 55 mg dose and for the F2 formulation (compared to 0.2 for Promacta® 50 mg dose); Ratio of 0.7 for the 75 mg dose (compared to 0.3 for Promacta® 75 mg dose).

### Example 9

### Food Effect Study in Health Volunteers

The effect of food, high and low in calcium content, on the pharmacokinetics of eltrombopag after administration of capsule comprising (80% w/w vitamin E TPGS and 20% w/w of eltrombopag olamine) will be investigated. The treatment will consist of single oral doses administered in a fasted state, and in various fed conditions: high-fat high-calorie (HFHC) high-calcium meal, HFHC low-calcium meal, high-fat low-calorie (HFLC) low-calcium meal. Subjects will undergo 4 treatment periods with a washout of 7 to 10 days between 2 consecutive doses. The primary objective is to evaluate the effect of food, high or low in calcium, on eltrombopag pharmacokinetics, including but not limited to the measure of AUC, Tmax and Cmax.

1. A pharmaceutical composition in an oral dosage form comprising 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid (eltrombopag) or a pharmaceutically acceptable salt thereof and vitamin E TPGS.

2. (canceled)

3. The pharmaceutical composition of claim 1, wherein the weight of eltrombopag, calculated in its form of free acid, is not more than 50% of the total weight of eltrombopag and vitamin E TPGS.

4-6. (canceled)

7. The pharmaceutical composition of claim 1 further comprising at least one anti-oxidant.

8. The pharmaceutical composition according to claim 3, wherein said at least one anti-oxidant is selected from a list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture thereof.

9-13. (canceled)

14. A pharmaceutical composition in an oral dosage form comprising 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid (eltrombopag) or a pharmaceutically acceptable salt thereof and at least one micelle forming agent.

15. (canceled)

16. The pharmaceutical composition according to claim 14, wherein the weight of eltrombopag is not more than 50% of the total weight of eltrombopag and the at least one micelle forming agent.

17-18. (canceled)

19. The pharmaceutical composition in an oral dosage form according to claim 14, wherein the at least one micelle forming agent is at least one surfactant.

20. The pharmaceutical composition of claim 19, wherein the at least one surfactant is anon-ionic surfactant.

21. The pharmaceutical composition according to claim 20, wherein the at least one surfactant is selected from the list consisting of Vitamin ETPGS, PEG 40 hydrogenated castor oil (Cremophor RH 40 or Kolliphor RH40), PEG 15 hydroxystearate (Solutol HS 15), PEG 32 monostearate (Gelucire 48/16), Gelucire 44/14, Gelucire 50/13, labrasol, PEG 35 castor oil (Cremophor EL) and Polyoxyethylene (20) sorbitan monooleate (Polysorbate 80, Tween 80), or a mixture thereof.

22-26. (canceled)

27. The pharmaceutical composition of claim 14, wherein the at least one micelle forming agent is a phospholipid.

28. (canceled)

29. The pharmaceutical composition of claim 27, wherein the phospholipids is diacyl-phospholipids.

30-31. (canceled)

32. The pharmaceutical composition of claim 27, wherein the phospholipids is monoacyl-phospholipids.

33. The pharmaceutical composition of any one of the claim 27, further comprising at least one bile salt.

34. (canceled)

35. The pharmaceutical composition of claim 27, wherein the weight of eltrombopag, calculated in its free acid form, is between about 5% to about 30% of the total weight of the pharmaceutical composition.

36. The pharmaceutical composition of claim 14 further comprising at least one anti-oxidant.

37. The pharmaceutical composition according to claim 36, wherein said at least one anti-oxidant is selected from a list consisting of a list consisting of Vitamin E, Butylhydroxytoluol (BHT), Butylhydroxyanisol (BHA), Propyl gallate, ascorbyl palmitate, ascorbic acid, EDTA and sodium metabisulfite or a mixture thereof.

38. (canceled)

39. The pharmaceutical composition according to claim 37 in the form of capsule.

40-47. (canceled)

48. A process for preparing the pharmaceutical composition according to any one of the claims 1-13, comprising the steps of:
a) Melting vitamin E TPGS, preferably by heating above its melting temperature;
b) Optionally adding an anti-oxidant, e.g. EDTA to the molten mass and mixing thoroughly
c) Adding eltrombopag or a pharmaceutically acceptable salt thereof to the molten mass and stirring to mix thoroughly;
d) Filling mixture c) into a capsule, suitably a HPMC capsule; and
e) Optionally seal the capsule by banding.

49. A method of measuring a drug dissolution in the presence or absence of coordinating metal comprising the steps of
a) Preparing a medium comprising a buffering system, a bile salt and phospholipids, wherein the resulting pH is about 6 to 8, about 6.5 to 7.5, preferably about 6.8;
b) Adding excessive amount of coordinating metals; preferably coordinating metal is calcium, aluminium or magnesium, preferably coordinating metal is calcium;
c) Optionally waiting for the excessive amount of coordinating metals to completely dissolve in the medium or to saturate in the medium;
d) adding the drug, preferably formulated in a formulation, preferably in a dosage form, into the medium; preferably said drug is eltrombopag, preferably said formulation is the pharmaceutical formulation of the invention typically comprising phospholipids or comprising at least one surfactant, preferably said dosage form is capsule or tablet;
e) Periodically taking solution out in the amount that is sufficient for measuring the dissolved drug concentration; preferably periodically refers to every 15 minutes, preferably every 15 minutes for at least the first hour, preferably after the addition of the drug;
f) Measuring the drug concentration.

50. The method of claim 49, wherein the drug is eltrombopag.
